(19) United States
(12) Patent Application Publication
(10) Pub. No.: US 2025/0179175 A1
(43) Pub. Date: Jun. 5, 2025

(54) ANTI-CD160 TRANSMEMBRANE ISOFORM ANTIBODIES

(71) Applicants: ALDERAAN BIOTECHNOLOGY, Paris (FR); INSTITUT NATIONAL DE LA SANTÉ ET DE LA RECHERCHE MÉDICALE, Paris (FR); UNIVERSITE PARIS EST CRETEIL VAL DE MARNE, Creteil Cedex (FR); UNIVERSITE DE REIMS CHAMPAGNE-ARDENNE, Reims Cedex (FR); UNIVERSITE PARIS CITE, Paris (FR); CENTRE NATIONAL DE LA RECHERCHE SCIENTIFIQUE, Paris (FR)

(72) Inventors: Arnaud FOUSSAT, Paris (FR); Armand BENSUSSAN, Paris Cedex 10 (FR); Anne MARIE-CARDINE, Paris Cedex 10 (FR); Jérôme GIUSTINIANI, Créteil (FR); Stéphane POTTEAUX, Paris Cedex 10 (FR); Jérôme DEVY, Reims Cedex (FR)

(21) Appl. No.: 18/843,754

(22) PCT Filed: Mar. 9, 2023
(86) PCT No.: PCT/EP2023/056014

§ 371 Date: Sep. 4, 2024

(30) Foreign Application Priority Data
Mar. 9, 2022 (EP) 22305265.5

(51) Int. Cl. C07K 16/28; A61K 39/00; A61P 35/00; A61P 37/02; G01N 33/577; G01N 33/68

(52) U.S. Cl. CPC C07K 16/2803; A61P 35/00; A61P 37/02; G01N 33/577; G01N 33/6872; A61K 2039/505; C07K 2317/24; C07K 2317/31; C07K 2317/565; C07K 2317/732; C07K 2317/75; C07K 2319/03; G01N 2333/70503

(57) ABSTRACT

The present invention relates to antibodies and antigen-binding fragments thereof, binding to CD160-TM isoform, and their use for treating cancer and other diseases.

### FIELD OF INVENTION

The present invention relates to antibodies and antigen-binding fragments thereof, binding to the CD160-TM transmembrane isoform and their use for treating cancer and other diseases.

### BACKGROUND OF INVENTION

CD160 has been initially identified as a GPI-anchored (CD160-GPI) MHC-class I activating receptor mainly expressed on peripheral blood NK cells. It was additionally reported the identification of a CD160 transmembrane isoform (CD160-TM) resulting from the alternative splicing of CD160 gene. It was established that CD160-TM surface expression is highly restricted to NK cells and is activation-dependent (Giustiniani J et al. J Immunol. 2009 Jan. 1; 182(1):63-71). Indeed, it is accepted in the art that CD160-TM is only expressed by activated NK cells, whereas CD160-GPI is expressed by NK cells (activated or not) and by different subsets of T cells. In addition, it was provided evidences that CD160-TM represent a novel activating receptor, as assessed by the increased CD107a NK cell surface mobilization observed upon its engagement (Giustiniani J et al. 2009).

Accordingly, antibodies that bind to the CD160-TM isoform without binding to the CD160 GPI-anchored isoform (nor to the CD160 soluble isoform that may result from the proteolytic cleavage of the CD160-GPI isoform) can thus be suitable for therapeutic purposes, and may present the advantage of avoiding systemic toxicity such as cytokine storm risk. For example, such an antibody may be used for amplifying NK cell activation and therefore effector functions of NK cells (cytotoxicity, cytokine secretion etc.) or for inducing depletion of CD160-TM expressing cells (in particular activated NK cells) in vivo.

In the present invention, the Inventors have developed new antibodies binding specifically to the CD160-TM isoform, that may be used as a medicament for human therapy.

### SUMMARY

The present invention relates to an isolated anti-human CD160-TM antibody or antigen-binding fragment thereof, wherein the variable region of the light chain (VL) of said antibody or antigen-binding fragment comprises the following complementary-determining region 3 (CDR3): X₁QSX₂SYPX₃T (SEQ ID NO: 15) wherein X₁ is G or L, X₂ is Y or Q, and X₃ is Y or F.

In one embodiment, the variable region of the heavy chain (VH) of the isolated anti-human CD160-TM antibody or antigen-binding fragment thereof as described hereinabove comprises the three following complementary-determining regions (CDRs):

CDR1:
(SEQ ID NO: 1)
GYTFTSYW,
CDR2:
(SEQ ID NO: 2)
IYPGSGFT,
CDR3:
(SEQ ID NO: 3)
SLDEGFAY;

the variable region of the light chain (VL) comprises the three following CDRs:

CDR1:
(SEQ ID NO: 4)
QGINIW,
CDR2:
KAS,
CDR3:
(SEQ ID NO: 5)
LQSQSYPFT

In one embodiment, the variable region of the heavy chain (VH) of the isolated anti-human CD160-TM antibody or antigen-binding fragment thereof as described hereinabove comprises the three following complementary-determining regions (CDRs):

CDR1:
(SEQ ID NO: 6)
GFSLTSYG,
CDR2:
(SEQ ID NO: 7)
IWRGGNT,
CDR3:
(SEQ ID NO: 8)
VKNGGNSKDYALDY;

the variable region of the light chain (VL) comprises the three following CDRs:

CDR1:
(SEQ ID NO: 9)
ENVGIY,
CDR2:
GSS,
CDR3:
(SEQ ID NO: 10)
GQSYSYPYT.

In one embodiment, said antibody or antigen-binding fragment comprises a heavy chain variable region comprising a sequence having at least 70% of identity with SEQ ID NO: 11 and a light chain variable region comprising a sequence having at least 70% of identity with SEQ ID NO: 12.

In one embodiment, said antibody or antigen-binding fragment comprises a heavy chain variable region comprising a sequence having at least 70% of identity with SEQ ID NO: 13 and a light chain comprising a sequence having at least 70% of identity with SEQ ID NO: 14.

In one embodiment, the isolated anti-human CD160-TM antibody or antigen-binding fragment thereof as described hereinabove is chimeric or humanized, preferably said antibody or antigen-binding fragment thereof is monoclonal.

In one embodiment, the isolated anti-human CD160-TM antibody or antigen-binding fragment thereof as described hereinabove is a bispecific antibody.

In one embodiment, the isolated anti-human CD160-TM antibody or antigen-binding fragment thereof as described hereinabove is conjugated, preferably said antibody or fragment thereof is conjugated to a detectable label.

The present invention further relates to a fusion protein comprising the isolated anti-human CD160-TM antibody or the antigen-binding fragment thereof as described hereinabove.

The present invention further relates to a nucleic acid encoding the isolated anti-human CD160-TM antibody or antigen-binding fragment thereof or a fusion protein as described hereinabove.

The present invention further relates to a pharmaceutical composition comprising the isolated anti-human CD160-TM antibody or antigen-binding fragment thereof or the fusion protein according as described hereinabove, and at least one pharmaceutically acceptable excipient.

The present invention further relates to the isolated anti-human CD160-TM antibody or antigen-binding fragment thereof, the fusion protein or the pharmaceutical composition as described hereinabove, for use as a medicament.

The present invention further relates to the isolated anti-human CD160-TM antibody or antigen-binding fragment thereof, the fusion protein or the pharmaceutical composition as described hereinabove for use in treating a cancer, an infectious disease, an autoimmune disease, an inflammatory disease or Paroxysmal Nocturnal Hemoglobinuria in a subject in need thereof.

In one embodiment, the isolated anti-human CD160-TM antibody or antigen-binding fragment thereof, the fusion protein or the pharmaceutical composition as described hereinabove is for use in treating a breast cancer in a subject in need thereof. In one embodiment, the isolated anti-human CD160-TM antibody or antigen-binding fragment thereof, the fusion protein or the pharmaceutical composition as described hereinabove is for use in treating a breast cancer selected from the group comprising or consisting of luminal A breast cancer, luminal B breast cancer, triple-negative breast cancer (TNBC) and HER2-positive breast cancer.

In one embodiment, the isolated anti-human CD160-TM antibody or antigen-binding fragment thereof, the fusion protein or the pharmaceutical composition as described hereinabove is for use in treating a NK leukemia or a NK lymphoma, such as for example, extranodal and non-extranodal NK/T lymphomas; NK cell derived malignancies; acute NK leukemia, and peripheral T-cell lymphoma (PTCL), such as for example peripheral T-cell lymphoma not otherwise specified (PTCL-NOS).

The present invention further relates to an in vitro method for detecting CD160-TM in a sample, preferably in a biological sample, comprising a step of contacting the sample with at least one isolated anti-human CD160-TM antibody or antigen-binding fragment thereof as described hereinabove.

The present invention further relates to the isolated anti-human CD160-TM antibody or antigen-binding fragment thereof as described hereinabove for use in the in vivo detection of tumors expressing CD160-TM in a subject.

### Definitions

In the present invention, the following terms have the following meanings:

“About”, preceding a figure encompasses plus or minus 10%, or less, of the value of said figure. It is to be understood that the value to which the term “about” refers is itself also specifically, and preferably, disclosed.

“Affinity”, as used herein, means the strength of the binding of an antibody to an epitope. The affinity of an antibody is given by the dissociation constant K_{D}, defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant K_{A} is defined by 1/K_{D}. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. Binding properties of an antibody or antigen-binding fragment thereof to antigens, cells or tissues may generally be determined and assessed using immunodetection methods including, for example, ELISA, immunofluorescence-based assays, such as immuno-histochemistry (IHC) and/or fluorescence-activated cell sorting (FACS) or by surface plasmon resonance (SPR, e.g., using BIAcore®).

“Antibody” and “immunoglobulin”, as used herein, may be used interchangeably and refer to a protein having a combination of two heavy and two light chains whether or not it possesses any relevant specific immunoreactivity. “Antibodies” refers to such assemblies which have significant known specific immunoreactive activity to an antigen of interest (e.g., CD160-transmembrane (TM) isoform). The term “anti-CD160-TM isoform” is used herein to refer to antibodies which exhibit immunological specificity for human CD160-TM isoform. As explained elsewhere herein, “specificity” for human CD160-TM isoform does not exclude cross-reaction with species homologues of CD160-TM isoform, such as, for example, with simian CD160-TM isoform. Antibodies and immunoglobulins comprise light and heavy chains, with or without an interchain covalent linkage between them. Basic immunoglobulin structures in vertebrate systems are relatively well understood. The generic term “immunoglobulin” comprises five distinct classes of antibody that can be distinguished biochemically. Although the following discussion will generally be directed to the IgG class of immunoglobulin molecules, all five classes of antibodies are within the scope of the present invention. With regard to IgG, immunoglobulins comprise two identical light polypeptide chains of molecular weight of about 23 kDa, and two identical heavy chains of molecular weight of about 53-70 kDa. The four chains are joined by disulfide bonds in a “Y” configuration wherein the light chains bracket the heavy chains starting at the mouth of the “Y” and continuing through the variable region. The light chains of an antibody are classified as either kappa (κ) or lambda (λ). Each heavy chain class may be bonded with either a κ or λ light chain. In general, the light and heavy chains are covalently bonded to each other, and the “tail” regions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma (γ), mu (μ), alpha (α), delta (δ) or epsilon (ε) with some subclasses among them (e.g., γ1-γ4). It is the nature of this chain that determines the “class” of the antibody as IgG, IgM, IgA IgD or IgE, respectively. The immunoglobulin subclasses or “isotypes” (e.g., IgG1, IgG2, IgG3, IgG4, IgA1, etc.) are well characterized and are known to confer functional specialization. Modified versions of each of these classes and isotypes are readily discernable to the skilled artisan in view of the instant disclosure and, accordingly, are within the scope of the present invention. As indicated above, the variable region of an antibody allows the antibody to selectively recognize and specifically bind epitopes on antigens. That is, the light chain variable domain (VL domain) and heavy chain variable domain (VH domain) of an antibody combine to form the variable region that defines a three-dimensional antigen binding site. This quaternary antibody structure forms the antigen binding site presents at the end of each arm of the “Y”. More specifically, the antigen binding site is defined by three complementarity determining regions (CDRs) on each of the VH and VL chains.

“Antigen-binding fragment”, as used herein, refers to a part or region of an antibody which comprises fewer amino acid residues than the whole antibody. An “antigen-binding fragment” binds antigen and/or competes with the whole antibody from which it derives for antigen binding (e.g., specific binding to CD160-TM isoform). Antigen-binding fragments encompasses, without any limitation, a single chain antibody, a dimeric single chain antibody, a Fv, a scFv, a Fab, a Fab′, a Fab′-SH, a F(ab)′2, a Fd, a defucosylated antibody, a diabody, a triabody and a tetrabody. It may also encompass a unibody, a domain antibody, and a nanobody.

“CD160” has its general meaning in the art and refers to CD160 molecule. Three CD160 isoforms exist: the CD160-TM isoform, the CD160 GPI-anchored isoform and the soluble CD160 isoform. CD160-GPI is expressed by intestinal intraepithelial T lymphocytes and by a minor subset of circulating lymphocytes including NK cells, TCRγδ and cytotoxic effector CD8^{bright}CD28-T lymphocytes (ANUMANTHAN et al., 1998, J Immunol; 161:2780-2790; MAIZA et al., J. Exp. Med., vol. 178, p: 1121-1126, 1993). The CD160 transmembrane isoform (“CD160-TM”) is described in Giustiniani J et al. (J Immunol. 2009 Jan. 1; 182(1):63-71.) as well as in the international patent application WO2008155363 and is characterized by the amino acid sequence as set forth in SEQ ID NO: 21. The extracellular domain of the CD160-TM isoform may be defined by the amino acid sequence ranging from the amino acid residue at position 26 to the amino acid residue at position 189 in SEQ ID NO: 21. The CD160 GPI-anchored isoform (“CD160-GPI”) is described in Nikolova M. et al. (Int Immunol. 2002 May; 14(5):445-51.) as well as in the international patent application WO2006015886 and is characterized by the amino acid sequence as set forth in SEQ ID NO: 22 fused to a GPI anchor at the C terminus end. The CD160 soluble isoform is described in Giustiniani J. et al. (J Immunol. 2007 Feb. 1; 178(3):1293-300) and is characterized by the amino acid sequence as set forth in SEQ ID NO: 22. In SEQ ID NO: 21-22, amino acids 1-25 correspond to a signal peptide, and may consequently be absent from the expressed protein.

SEQ ID NO: 21: CD160-TM isoform
MLLEPGRGCCALAILLAIVDIQSGGCINITSSASQEGTRLNLICTVWHK
KEEAEGFVVFLCKDRSGDCSPETSLKQLRLKRDPGIDGVGEISSQLMFT
ISQVTPLHSGTYQCCARSQKSGIRLQGHFFSILFTETGNYTVTGLKQRQ
HLEFSHNEGTLSSGFLQEKVWVMLVTSLVALQGMSKRAVSTPSNEGAII
FLPPWLFSRRRRLERMSRGREKCYSSPGYPQESSNQFH
SEQ ID NO: 22: CD160 soluble isoform
MLLEPGRGCCALAILLAIVDIQSGGCINITSSASQEGTRLNLICTVWHK
KEEAEGFVVFLCKDRSGDCSPETSLKQLRLKRDPGIDGVGEISSQLMFT
ISQVTPLHSGTYQCCARSQKSGIRLQGHFFSILFTETGNYTVTGLKQRQ
HLEFSHNEGTLSS

“CDR” or “complementarity determining region” means the non-contiguous antigen combining sites found within the variable region of both heavy and light chain polypeptides. The precise amino acid sequence boundaries of a given CDR can be determined using any of a number of well-known schemes, including those described by Kabat et al. (1991), “Sequences of Proteins of Immunological Interest” 5th Ed. Public Health Service, National Institutes of Health, Bethesda, MD (“Kabat” numbering scheme), Al-Lazikani et al., (1997) JMB 273,927-948 (“Chothia” numbering scheme), or a combination thereof, or ImMunoGeneTics (IMGT) Information System® (Lefranc et al., Nucleic Acids Res. 27: 209-212 1999). IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues may be readily identified. This information can be used in grafting and replacement of CDR residues from immunoglobulins of one species into an acceptor framework from, typically, a human antibody. Correspondence between the Kabat numbering and the IMGT unique numbering system is also well known to one skilled in the art (e.g., Lefranc et al., supra). Thus, in one embodiment, by CDR regions or CDR, it is intended to indicate the hypervariable regions of the heavy and light chains of the immunoglobulins as defined by IMGT® numbering system.

“Diabodies”, as used herein, refer to small antibody fragments prepared by constructing scFv fragments with short linkers (about 5-10 residues) between the VH and VL such that inter-chain but not intra-chain pairing of the variable domains is achieved, resulting in a bivalent fragment, i.e., fragment having two antigen-binding sites. Bispecific diabodies are heterodimers of two “crossover” scFv fragments in which the VH and VL of the two antibodies are present on different polypeptide chains. Diabodies are described, for example, in patent EP0404097 or patent application WO1993011161.

“Domain antibodies” refer to the smallest functional binding units of antibodies, corresponding to the variable regions of either the heavy or light chains of antibodies.

“Epitope” refers to a specific arrangement of amino acids located on a protein or proteins to which an antibody or antigen-binding fragment thereof binds. Epitopes often consist of a chemically active surface grouping of molecules such as amino acids or sugar side chains, and have specific three-dimensional structural characteristics as well as specific charge characteristics. Epitopes can be linear (or sequential) or conformational, i.e., involving two or more sequences of amino acids in various regions of the antigen that may not necessarily be contiguous.

“Fab” refers to fragment antibodies generated by papain digestion of whole IgG antibodies to remove the entire Fc fragment, including the hinge region. These antibodies are monovalent, containing only a single antigen binding site. In contrast, F(ab′)₂ fragment antibodies are generated by pepsin digestion of whole IgG antibodies to remove most of the Fc region while leaving intact some of the hinge region. F(ab′)2 fragments have two antigen-binding F(ab) portions linked together by disulfide bonds. The term “Fab′” refers to an antibody fragment having a molecular weight of about 50,000 and antigen binding activity, which is obtained by cutting a disulfide bond of the hinge region of the F(ab′)₂. Fab′-SH is the designation herein for Fab′ in which the cysteine residue(s) of the constant domains bear a free thiol group.

“Fc domain,” “Fc portion,” and “Fc region” refer to a C-terminal fragment of an antibody heavy chain, e.g., from about amino acid (aa) 230 to about aa 450 of human gamma heavy chain or its counterpart sequence in other types of antibody heavy chains (e.g., α, δ, ε and μ for human antibodies), or a naturally occurring allotype thereof.

“Fd fragment” refers to the heavy chain of the Fab fragment, comprising the VH and CH1 regions.

“Framework region” or “FR region” includes the amino acid residues that are part of the variable region, but are not part of the CDRs (e.g., using the IMGT® numbering definition of CDRs). The framework regions for the light chain are similarly separated by each of the VL's CDRs. In naturally occurring antibodies, the six CDRs present on each monomeric antibody are short, non-contiguous sequences of amino acids that are specifically positioned to form the antigen binding site as the antibody assumes its three-dimensional configuration in an aqueous environment. The remainders of the heavy and light variable domains show less inter-molecular variability in amino acid sequence and are termed the framework regions. The framework regions largely adopt a β-sheet conformation and the CDRs form loops which connect, and in some cases form part of, the 3-sheet structure. Thus, these framework regions act to form a scaffold that provides for positioning the six CDRs in correct orientation by inter-chain, non-covalent interactions. The antigen binding site formed by the positioned CDRs defines a surface complementary to the epitope on the immunoreactive antigen. This complementary surface promotes the non-covalent binding of the antibody to the immunoreactive antigen epitope. The position of CDRs can be readily identified by one of ordinary skill in the art.

“Fv”, as used herein, refers to the minimum antibody fragment that contains a complete antigen-recognition and -binding site. This fragment consists of a dimer of one VH and one VL in tight, non-covalent association. From the folding of these two domains emanate six hypervariable loops (three loops each from the heavy and light chain) that contribute to antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Heavy chain region” includes amino acid sequences derived from the constant domains of an immunoglobulin heavy chain. A protein comprising a heavy chain region comprises at least one of a C_{H}1 domain, a hinge (e.g., upper, middle, and/or lower hinge region) domain, a C_{H}2 domain, a C_{H}3 domain, or a variant or fragment thereof. In an embodiment, the antibody or antigen-binding fragment thereof according to the present invention may comprise the Fc region of an immunoglobulin heavy chain (e.g., a hinge portion, a C_{H}2 domain, and a C_{H}3 domain). In another embodiment, the antibody or antigen-binding fragment thereof according to the present invention lacks at least a region of a constant domain (e.g., all or part of a C_{H}2 domain). In certain embodiments, at least one, and preferably all, of the constant domains is or are derived from a human immunoglobulin heavy chain. For example, in one embodiment, the heavy chain region comprises a fully human hinge domain. In other embodiments, the heavy chain region comprises a fully human Fc region (e.g., hinge, C_{H}2 and C_{H}3 domain sequences from a human immunoglobulin). In certain embodiments, the constituent constant domains of the heavy chain region are from different immunoglobulin molecules. For example, a heavy chain region of a protein may comprise a C_{H}2 domain derived from an IgG1 molecule and a hinge region derived from an IgG3 or IgG4 molecule. In other embodiments, the constant domains are chimeric domains comprising regions of different immunoglobulin molecules. For example, a hinge may comprise a first region from an IgG1 molecule and a second region from an IgG3 or IgG4 molecule. In some embodiments, the constant domains of the heavy chain region may be modified such that they vary in amino acid sequence from the naturally occurring (wild-type) immunoglobulin molecule. That is, the antibody or antigen-binding fragment thereof according to the present invention may comprise alterations or modifications to one or more of the heavy chain constant domains (C_{H}1, hinge, C_{H}2 or C_{H}3) and/or to the light chain constant domain (C_{L}). Exemplary modifications include additions, deletions or substitutions of one or more amino acids in one or more domains.

“Hinge region” includes the region of a heavy chain molecule that joins the C_{H}1 domain to the C_{H}2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., 1998. J Immunol. 161(8):4083-90).

“Identity” or “identical”, when used herein in a relationship between the sequences of two or more amino acid sequences, or of two or more nucleic acid sequences, refers to the degree of sequence relatedness between amino acid sequences or nucleic acid sequences, as determined by the number of matches between strings of two or more amino acid residues or nucleic acid residues. “Identity” measures the percent of identical matches between the smaller of two or more sequences with gap alignments (if any) addressed by a particular mathematical model or computer program (i.e., “algorithms”). Identity of related amino acid sequences or nucleic acid sequences can be readily calculated by known methods. Such methods include, but are not limited to, those described in Lesk A. M. (1988). Computational molecular biology: Sources and methods for sequence analysis. New York, NY: Oxford University Press; Smith D. W. (1993). Biocomputing: Informatics and genome projects. San Diego, CA: Academic Press; Griffin A. M. & Griffin H. G. (1994). Computer analysis of sequence data, Part 1. Totowa, NJ: Humana Press; von Heijne G. (1987). Sequence analysis in molecular biology: treasure trove or trivial pursuit. San Diego, CA: Academic press; Gribskov M. R. & Devereux J. (1991). Sequence analysis primer. New York, NY: Stockton Press; Carillo et al., 1988. SIAM J Appl Math. 48(5):1073-82. Preferred methods for determining identity are designed to give the largest match between the sequences tested. Methods of determining identity are described in publicly available computer programs. Preferred computer program methods for determining identity between two sequences include the GCG program package, including GAP (Genetics Computer Group, University of Wisconsin, Madison, WI; Devereux et al., 1984. Nucleic Acids Res. 12(1 Pt 1):387-95), BLASTP, BLASTN, and FASTA (Altschul et al., 1990. J Mol Biol. 215(3):403-10). The BLASTX program is publicly available from the National Center for Biotechnology Information (NCBI) and other sources (BLAST Manual, Altschul et al. NCB/NLM/NIH Bethesda, Md. 20894). The well-known Smith Waterman algorithm may also be used to determine identity.

“Isolated protein”, and in particular “isolated antibody”, as used herein, is intended to refer to a protein, in particular an antibody that is substantially free of other proteins or antibodies having different antigenic specificities (e.g., an isolated protein or antibody that specifically binds CD160-TM isoform is substantially free of proteins or antibodies that specifically bind antigens other than CD160-TM isoform). An isolated protein, in particular an isolated antibody, that specifically binds CD160-TM isoform may, however, have cross-reactivity to other antigens, such as CD160-TM molecules from other species. Moreover, an isolated protein or antibody may be substantially free of other cellular material and/or chemicals, in particular those that would interfere with therapeutic uses of the protein or antibody, including without limitation, enzymes, hormones, and other proteinaceous or non-proteinaceous components.

“Mammal” refers to any mammal, including humans, non-human primates, domestic and farm animals, and zoo, sports, or pet animals, such as dogs, cats, cattle, horses, sheep, pigs, goats, rabbits, monkeys, etc. Preferably, the mammal is human.

“Monoclonal antibody” refers to an antibody obtained from a population of substantially homogeneous antibodies, i.e., the individual antibodies comprised in the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies or antigen-binding fragment thereof according to the present invention may be prepared by the hybridoma methodology first described by Kohler et al., 1975. Nature. 256(5517):495-7, or may be made using recombinant DNA methods in bacterial, eukaryotic animal or plant cells (U.S. Pat. No. 4,816,567). The “monoclonal antibodies” may also be isolated from phage antibody libraries using the techniques described in Clackson et al., 1991. Nature. 352(6336):624-8 and Marks et al., 1991. J Mol Biol. 222(3):581-97, for example.

“Nanobodies” refer to antibody-derived therapeutic proteins that contain the unique structural and functional properties of naturally-occurring heavy chain antibodies (Muyldermans, 2013. Annu Rev Biochem. 82:775-97). These heavy chain antibodies may contain a single variable domain (VHH) and two constant domains (C_{H}2 and C_{H}3).

“Single chain antibody”, as used herein, refers to any antibody or fragment thereof that is a protein having a primary structure comprising or consisting of one uninterrupted sequence of contiguous amino acid residues, including without limitation (1) single-chain Fv molecules (scFv); (2) single chain proteins containing only one light chain variable domain, or a fragment thereof that contains the three CDRs of the light chain variable domain, without an associated heavy chain moiety; and (3) single chain proteins containing only one heavy chain variable region, or a fragment thereof containing the three CDRs of the heavy chain variable region, without an associated light chain moiety.

“Single-chain Fv”, also abbreviated as “sFv” or “scFv”, refers to antibody fragments that comprise the V_{H} and V_{L} antibody domains connected into a single amino acid chain. Preferably, the scFv amino acid sequence further comprises a peptide linker between the V_{H} and V_{L} domains that enables the scFv to form the desired structure for antigen binding.

“Specificity” refers to the ability of an antibody to detectably bind an epitope presented on an antigen, such as a CD160-TM, while having relatively little detectable reactivity with non-CD160-TM proteins such as the CD160 GPI-anchored isoform and the CD160 soluble isoform. Specificity can be relatively determined by binding or competitive binding assays, using, e.g., Biacore instruments. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules (in this case the specific antigen is a CD160-TM polypeptide).

“Subject” refers to a warm-blooded animal, preferably a mammal (including humans, domestic and farm animals, and zoo, sports, or pet animals, such as dogs, cats, cattle, horses, sheep, pigs, goats, rabbits, etc . . . ), and more preferably a human. In one embodiment, a subject may be a “patient”, i.e., a warm-blooded animal, more preferably a human, who/which is awaiting the receipt of, or is receiving medical care or was/is/will be the object of a medical procedure, or is monitored for the development of a disease. In one embodiment, the subject is an adult (for example a subject above the age of 18). In another embodiment, the subject is a child (for example a subject below the age of 18). In one embodiment, the subject is a male. In another embodiment, the subject is a female.

“Therapeutically effective amount” refers to the level or amount of an antibody or antigen-binding fragment thereof as described herein that is aimed at, without causing significant negative or adverse side effects to the target, (1) delaying or preventing the onset of a disease, disorder, or condition; (2) slowing down or stopping the progression, aggravation, or deterioration of one or more symptoms of the disease, disorder, or condition; (3) bringing about ameliorations of the symptoms of the disease, disorder, or condition; (4) reducing the severity or incidence of the disease, disorder, or condition; or (5) curing the disease, disorder, or condition. A therapeutically effective amount may be administered prior to the onset of the disease, disorder, or condition, for a prophylactic or preventive action. Alternatively or additionally, the therapeutically effective amount may be administered after initiation of the disease, disorder, or condition, for a therapeutic action.

“Treating” or “treatment” or “alleviation” refers to both therapeutic treatment and prophylactic or preventative measures; wherein the object is to prevent or slow down (lessen) the targeted pathologic condition or disorder. Those in need of treatment include those already with the disorder as well as those prone to have the disorder or those in whom the disorder is to be prevented. In one embodiment, a subject is successfully “treated” for a disease as described herein if, after receiving a therapeutic amount of an antibody or antigen-binding fragment thereof, a fusion protein, a nucleic acid or an expression vector as described herein, the subject shows at least one of the following: reduction in the number of pathogenic cells; relief to some extent of one or more of the symptoms associated with the disease to be treated; reduced morbidity and mortality; and improvement in quality of life issues. The above parameters for assessing successful treatment and improvement in the disease are readily measurable by routine procedures familiar to a physician.

“Unibodies” refer to an antibody fragment lacking the hinge region of IgG4 antibodies. The deletion of the hinge region results in a molecule that is essentially half the size of traditional IgG4 antibodies and has a univalent binding region rather than the bivalent biding region of IgG4 antibodies.

“Variable” refers to the fact that certain regions of the variable domains V_{H} and V_{L} differ extensively in sequence among antibodies and are used in the binding and specificity of each particular antibody for its target antigen. However, the variability is not evenly distributed throughout the variable domains of antibodies. It is concentrated in three segments called “hypervariable loops” in each of the V_{L} domain and the V_{H} domain which form part of the antigen binding site. The first, second and third hypervariable loops of the Vλ light chain domain are referred to herein as L1 (λ), L2 (λ) and L3 (λ) and may be defined as comprising residues 24-33 (L1(λ), consisting of 9, 10 or 11 amino acid residues), 49-53 L2 (λ), consisting of 3 residues) and 90-96 (L3(λ), consisting of 6 residues) in the V_{L} domain (Morea et al., 2000. Methods. 20(3):267-79). The first, second and third hypervariable loops of the Vκ light chain domain are referred to herein as L1(κ), L2(κ) and L3(κ) and may be defined as comprising residues 25-33 (L1(κ), consisting of 6, 7, 8, 11, 12 or 13 residues), 49-53 (L2(κ), consisting of 3 residues) and 90-97 (L3(κ), consisting of 6 residues) in the V_{L} domain (Morea et al., supra). The first, second and third hypervariable loops of the VH domain are referred to herein as H1, H2 and H3 and may be defined as comprising residues 25-33 (H1, consisting of 7, 8 or 9 residues), 52-56 (H2, consisting of 3 or 4 residues) and 91-105 (H3, highly variable in length) in the VH domain (Morea et al., supra). Unless otherwise indicated, the terms L1, L2 and L3 respectively refer to the first, second and third hypervariable loops of a VL domain, and encompass hypervariable loops obtained from both Vκ and Vλ isotypes. The terms H1, H2 and H3 respectively refer to the first, second and third hypervariable loops of the V_{H} domain, and encompass hypervariable loops obtained from any of the known heavy chain isotypes, including gamma (γ), mu (μ), alpha (α), delta (δ) or epsilon (ε). The hypervariable loops may each comprise part of a “complementarity determining region” or “CDR”, as defined hereinabove.

### DETAILED DESCRIPTION

The present invention relates to an antibody or antigen-binding fragment thereof which binds to CD160-TM isoform.

Examples of antibodies and antigen-binding fragments include, without limitation, a whole antibody, a single chain antibody, a dimeric single chain antibody, a Fv, a scFv, a Fab, a Fab′, a Fab′-SH, a F(ab)′₂, a Fd, a defucosylated antibody, a bispecific antibody, a diabody, a triabody, a tetrabody, a unibody, a domain antibody, and a nanobody.

In one embodiment, the antibody or antigen-binding fragment thereof according to the present invention is selected from the group comprising or consisting of a whole antibody, a single chain antibody, a dimeric single chain antibody, a Fv, a scFv, a Fab, a Fab′, a Fab′-SH, a F(ab)′₂, a Fd, a defucosylated antibody, a bispecific antibody, a diabody, a triabody and a tetrabody.

In one embodiment, the antibody or antigen-binding fragment thereof according to the present invention is selected from the group comprising or consisting of a whole antibody, a single chain variable fragment (scFv), a Fv, a Fab, a Fab′, a Fab′-SH, a F(ab)′₂, a defucosylated antibody, a bispecific antibody, a diabody, a triabody and a tetrabody.

In one embodiment, the isolated antibody or antigen-binding fragment according to the present invention is selected from the group comprising or consisting of a unibody, a domain antibody, and a nanobody.

In one embodiment, the isolated antibody or antigen-binding fragment according to the present invention is selected from the group comprising or consisting a whole antibody, a Fv, a scFv, a Fab, and a unibody.

In one embodiment, the antibody or the antigen-binding fragment thereof according to the present invention binds specifically to the CD160-TM isoform. In one embodiment, the antibody or the antigen-binding fragment thereof according to the present invention binds specifically to the human CD160-TM isoform.

As used herein, “binds specifically” refers to an antibody or an antigen-binding fragment thereof which recognizes and binds with a binding partner present in a sample, but which antibody or antigen-binding fragment thereof does not substantially recognize or bind other molecules in the sample.

In one embodiment, the antibody or the antigen-binding fragment thereof according to the present invention has low or no affinity for other CD160 isoforms, such as the CD160 GPI-anchored isoform and the CD160 soluble isoform.

Affinities of antibodies or antigen-binding fragments thereof can be readily determined using conventional techniques, for example, those described by Scatchard, 1949. Ann NY Acad Sci. 51:660-672. Binding properties of an antibody or an antigen-binding fragment thereof to antigens, cells or tissues may generally be determined and assessed using immunodetection methods including, for example, ELISA, immunofluorescence-based assays, such as immuno-histochemistry (IHC) and/or fluorescence-activated cell sorting (FACS) or by surface plasmon resonance (SPR, e.g., using BIAcore®).

In the following, and unless explicitly mentioned otherwise, CDR numbering and definitions are according to the IMGT® numbering system.

In one embodiment, the antibody or antigen-binding fragment thereof according to the present invention comprises a heavy chain variable region (abbreviated herein as VH) comprising the following complementary-determining region 1 (CDR1): GX₁X₂X₃TSYX₄ (SEQ ID NO: 16), wherein X₁ is F or Y, X₂ is S or T, X₃ is L or F, X₄ is G or W.

In one embodiment, the antibody or antigen-binding fragment thereof according to the present invention comprises a light chain variable region (abbreviated herein as VL) comprising the following complementary-determining region 3 (CDR3): X₁QSX₂SYPX₃T (SEQ ID NO: 15) wherein X₁ is G or L, X₂ is Y or Q, and X₃ is Y or F.

In one embodiment, the antibody or antigen-binding fragment thereof according to the present invention comprises a heavy chain variable region which comprises at least one, preferably at least two, more preferably the three following complementary-determining regions (CDRs):

CDR1:
(SEQ ID NO: 1)
GYTFTSYW;
CDR2:
(SEQ ID NO: 2)
IYPGSGFT;
and/or
CDR3:
(SEQ ID NO: 3)
SLDEGFAY.

In one embodiment, the antibody or antigen-binding fragment thereof according to the present invention comprises a heavy chain variable region which comprises at least one, preferably at least two, more preferably the three following complementary-determining regions (CDRs):

CDR1:
(SEQ ID NO: 6)
GFSLTSYG;
CDR2:
(SEQ ID NO: 7)
IWRGGNT;
and/or
CDR3:
(SEQ ID NO: 8)
VKNGGNSKDYALDY.

In one embodiment, any of CDR1, CDR2 and/or CDR3 of the VH with SEQ ID NOs 1-3, 6-8 can be characterized as having 1, 2, 3 or more amino acids being substituted by a different amino acid.

In one embodiment, any of CDR1, CDR2 and/or CDR3 of the VH with SEQ ID NOs 1-3, 6-8 can be characterized as having an amino acid sequence that shares at least 70%, 75%, 80%, 85%, 90%, 95%, 96%, 97%, 98%, 99% or more of identity with the particular CDR or sets of CDRs listed in the corresponding SEQ ID NOs.

In one embodiment, the antibody or antigen-binding fragment thereof according to the present invention comprises a light chain variable region which comprises at least one, preferably at least two, more preferably the three following complementary-determining regions (CDRs):

CDR1:
(SEQ ID NO: 4)
QGINIW;
CDR2:
KAS;
and/or
CDR3:
(SEQ ID NO: 5)
LQSQSYPFT.

In one embodiment, the antibody or antigen-binding fragment thereof according to the present invention comprises a light chain variable region which comprises at least one, preferably at least two, more preferably the three following complementary-determining regions (CDRs):

CDR1:
(SEQ ID NO: 9)
ENVGIY;
CDR2:
GSS;
and/or
CDR3:
(SEQ ID NO: 10)
GQSYSYPYT.

In one embodiment, any of CDR1, CDR2 and/or CDR3 of the VL with SEQ ID NO: 4 or 9, the sequence KAS or GSS, and SEQ ID NO: 5 or 10, respectively, can be characterized as having 1, 2, 3 or more amino acids being substituted by a different amino acid.

In one embodiment, any of CDR1, CDR2 and/or CDR3 of the VL with SEQ ID NO: 4 or 9, the sequence KAS or GSS, and SEQ ID NO: 5 or 10, respectively, can be characterized as having an amino acid sequence that shares at least 70%, 75%, 80%, 85%, 90%, 95%, 96%, 97%, 98%, 99% or more of identity with the particular CDR or sets of CDRs listed above.

In one embodiment, the antibody or antigen-binding fragment thereof according to the present invention comprises:
a VH which comprises at least one, preferably at least two, more preferably the three following CDRs:

CDR1:
(SEQ ID NO: 1)
GYTFTSYW;
CDR2:
(SEQ ID NO: 2)
IYPGSGFT;
and/or
CDR3:
(SEQ ID NO: 3)
SLDEGFAY;

and
a VL which comprises at least one, preferably at least two, more preferably the three following CDRs:

CDR1:
(SEQ ID NO: 4)
QGINIW;
CDR2:
KAS;
and/or
CDR3:
(SEQ ID NO: 5)
LQSQSYPFT.

In one embodiment, the antibody or antigen-binding fragment thereof according to the present invention comprises:
a VH which comprises the three following CDRs:

CDR1:
(SEQ ID NO: 1)
GYTFTSYW;
CDR2:
(SEQ ID NO: 2)
IYPGSGFT;
and
CDR3:
(SEQ ID NO: 3)
SLDEGFAY;

and
a VL which comprises the three following CDRs:

CDR1:
(SEQ ID NO: 4)
QGINIW;
CDR2:
KAS;
and
CDR3:
(SEQ ID NO: 5)
LQSQSYPFT.

In one embodiment, any of CDR1, CDR2 and/or CDR3 of the VH with SEQ ID NOs 1-3 and/or the VL with SEQ ID NO: 4, the sequence KAS and SEQ ID NO: 5, respectively, can be characterized as having 1, 2, 3 or more amino acids being substituted by a different amino acid.

In one embodiment, any of CDR1, CDR2 and/or CDR3 of the VH with SEQ ID NOs 1-3 and/or of the VL with SEQ ID NO: 4, the sequence KAS and SEQ ID NO: 5, respectively, can be characterized as having an amino acid sequence that shares at least 70%, 75%, 80%, 85%, 90%, 95%, 96%, 97%, 98%, 99% or more of identity with the particular CDR or sets of CDRs listed above.

An example of antibodies comprising a heavy chain comprising CDR1, CDR2 and CDR3 with SEQ ID NOs 1-3 and a light chain comprising CDR1, CDR2 and CDR3 with SEQ ID NO: 4, the sequence KAS and SEQ ID NO: 5, respectively, is 21C8 antibody.

In one embodiment, the antibody or antigen-binding fragment thereof according to the present invention comprises:
a VH which comprises at least one, preferably at least two, more preferably the three following CDRs:

CDR1:
(SEQ ID NO: 6)
GFSLTSYG;
CDR2:
(SEQ ID NO: 7)
IWRGGNT;
and/or
CDR3:
(SEQ ID NO: 8)
VKNGGNSKDYALDY;

a VL which comprises at least one, preferably at least two, more preferably the three following CDRs:

CDR1:
(SEQ ID NO: 9)
ENVGIY;
CDR2:
GSS;
and/or
CDR3:
(SEQ ID NO: 10)
GQSYSYPYT.

In one embodiment, the antibody or antigen-binding fragment thereof according to the present invention comprises:
a VH which comprises the three following CDRs:

CDR1:
(SEQ ID NO: 6)
GFSLTSYG;
CDR2:
(SEQ ID NO: 7)
IWRGGNT;
and
CDR3:
(SEQ ID NO: 8)
VKNGGNSKDYALDY;

and
a VL which comprises the three following CDRs:

CDR1:
(SEQ ID NO: 9)
ENVGIY;
CDR2:
GSS;
and
CDR3:
(SEQ ID NO: 10)
GQSYSYPYT.

In one embodiment, any of CDR1, CDR2 and/or CDR3 of the VH with SEQ ID NOs 6-8 and/or the VL with SEQ ID NO: 9, the sequence GSS and SEQ ID NO: 10, respectively, can be characterized as having 1, 2, 3 or more amino acids being substituted by a different amino acid.

In one embodiment, any of CDR1, CDR2 and/or CDR3 of the VH with SEQ ID NOs 6-8 and/or of the VL with SEQ ID NO: 9, the sequence GSS and SEQ ID NO: 10, respectively, can be characterized as having an amino acid sequence that shares at least 70%, 75%, 80%, 85%, 90%, 95%, 96%, 97%, 98%, 99% or more of identity with the particular CDR or sets of CDRs listed above.

An example of antibodies comprising a heavy chain comprising CDR1, CDR2 and CDR3 with SEQ ID NOs 6-8 and a light chain comprising CDR1, CDR2 and CDR3 with SEQ ID NO: 9, the sequence GSS and SEQ ID NO: 10, respectively, is 22B12 antibody.

In one embodiment, the antibody or antigen-binding fragment thereof according to the present invention comprises:
a VH which comprises at least one, preferably at least two, more preferably the three following CDRs:

CDR1:
(SEQ ID NO: 6)
GFSLTSYG;
CDR2:
(SEQ ID NO: 7)
IWRGGNT;
and/or
CDR3:
(SEQ ID NO: 8)
VKNGGNSKDYALDY;

and
a VL which comprises at least one, preferably at least two, more preferably the three following CDRs:

CDR1:
(SEQ ID NO: 4)
QGINIW;
CDR2:
KAS;
and/or
CDR3:
(SEQ ID NO: 5)
LQSQSYPFT.

In one embodiment, the antibody or antigen-binding fragment thereof according to the present invention comprises:
a VH which comprises the three following CDRs:

CDR1:
(SEQ ID NO: 6)
GFSLTSYG;
CDR2:
(SEQ ID NO: 7)
IWRGGNT;
and/or
CDR3:
(SEQ ID NO: 8)
VKNGGNSKDYALDY;

a VL which comprises the three following CDRs:

CDR1:
(SEQ ID NO: 4)
QGINIW;
CDR2:
KAS;
and/or
CDR3:
(SEQ ID NO: 5)
LQSQSYPFT.

In one embodiment, any of CDR1, CDR2 and/or CDR3 of the VH with SEQ ID NOs 6-8 and/or the VL with SEQ ID NO: 4, the sequence KAS and SEQ ID NO: 5, respectively, can be characterized as having 1, 2, 3 or more amino acids being substituted by a different amino acid.

In one embodiment, any of CDR1, CDR2 and/or CDR3 of the VH with SEQ ID NOs 6-8 and/or of the VL with SEQ ID NO: 4, the sequence KAS and SEQ ID NO: 5, respectively, can be characterized as having an amino acid sequence that shares at least 70%, 75%, 80%, 85%, 90%, 95%, 96%, 97%, 98%, 99% or more of identity with the particular CDR or sets of CDRs listed above.

In one embodiment, the antibody or antigen-binding fragment thereof according to the present invention comprises:
a VH which comprises at least one, preferably at least two, more preferably the three following CDRs:

CDR1:
(SEQ ID NO: 1)
GYTFTSYW;
CDR2:
(SEQ ID NO: 2)
IYPGSGFT;
and/or
CDR3:
(SEQ ID NO: 3)
SLDEGFAY;

and
a VL which comprises at least one, preferably at least two, more preferably the three following CDRs:

CDR1:
(SEQ ID NO: 9)
ENVGIY;
CDR2:
GSS;
and/or
CDR3:
(SEQ ID NO: 10)
GQSYSYPYT.

In one embodiment, the antibody or antigen-binding fragment thereof according to the present invention comprises:
a VH which comprises the three following CDRs:

CDR1:
(SEQ ID NO: 1)
GYTFTSYW;
CDR2:
(SEQ ID NO: 2)
IYPGSGFT;
and/or
CDR3:
(SEQ ID NO: 3)
SLDEGFAY;

and
a VL which comprises the three following CDRs:

CDR1:
(SEQ ID NO: 9)
ENVGIY;
CDR2:
GSS;
and/or
CDR3:
(SEQ ID NO: 10)
GQSYSYPYT.

In one embodiment, any of CDR1, CDR2 and/or CDR3 of the VH with SEQ ID NOs 1-3 and/or the VL with SEQ ID NO: 9, the sequence GSS and SEQ ID NO: 10, respectively, can be characterized as having 1, 2, 3 or more amino acids being substituted by a different amino acid.

In one embodiment, any of CDR1, CDR2 and/or CDR3 of the VH with SEQ ID NOs 1-3 and/or of the VL with SEQ ID NO: 9, the sequence GSS and SEQ ID NO: 10, respectively, can be characterized as having an amino acid sequence that shares at least 70%, 75%, 80%, 85%, 90%, 95%, 96%, 97%, 98%, 99% or more of identity with the particular CDR or sets of CDRs listed above.

In one embodiment, the antibody or antigen-binding fragment thereof according to the present invention comprises a VH comprising or consisting of the sequence SEQ ID NO: 11.

SEQ ID NO: 11
QVQLQQPGSELVRPGASVRLSCKASGYTFTSYWMHWVRQRHGQGLEWIG
NIYPGSGFTNYDEKFKNKGTLTVDTSSSTAYMHLSSLTSEDSAVYYCSL
DEGFAYWGQGTLVTVSA

In one embodiment, the antibody or antigen-binding fragment thereof according to the present invention comprises a VH comprising or consisting of the sequence SEQ ID NO: 13.

SEQ ID NO: 13
QVQLKQSGPGLVQPSQSLSIMCTVSGFSLTSYGLHWVRQSPGKGLEWLG
VIWRGGNTDYNADFMSRLTITKDNSKSQVFFKMNSLQADDTAVYYCVKN
GGNSKDYALDYWGQGTSVTVSS

In one embodiment, the VH comprises or consists of the sequence SEQ ID NO: 11 or 13 with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids substituted by a different amino acid.

In one embodiment, the VH has an amino acid sequence that shares at least 70%, 75%, 80%, 85%, 90%, 95%, 96%, 97%, 98%, 99% or more of identity with SEQ ID NO: 11 or 13.

In one embodiment, the antibody or antigen-binding fragment thereof according to the present invention comprises a VL comprising or consisting of the sequence SEQ ID NO: 12.

SEQ ID NO: 12
DIQMNQSPSSLSASLGDTITITCRASQGINIWLNWYQQKPGNIPKRLIY
KASNLHTGVPPRFRGSGSGTDFTLTISSLQPEDIATYYCLQSQSYPFTF
GSGTKLEIR

In one embodiment, the antibody or antigen-binding fragment thereof according to the present invention comprises a VL comprising or consisting of the sequence SEQ ID NO: 14.

SEQ ID NO: 14
NIVMTQSPKSMSMSVGERVTLSCKASENVGIYVSWYQQKPEQSPKLLIY
GSSNRYTGVPDRFTGSGSATDFTLIISSVQAEDLADYHCGQSYSYPYTF
GGGTKLEIK

In one embodiment, the VL comprises or consists of the sequence SEQ ID NO: 12 or 14 with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids substituted by a different amino acid.

In one embodiment, the VL has an amino acid sequence that shares at least 70%, 75%, 80%, 85%, 90%, 95%, 96%, 97%, 98%, 99% or more of identity with SEQ ID NO: 12 or 14.

In one embodiment, the antibody or antigen-binding fragment thereof according to the present invention comprises:
a VH comprising or consisting of the sequence SEQ ID NO: 11; and
a VL comprising or consisting of the sequence SEQ ID NO: 12.

In one embodiment, the VH comprises or consists of the sequence SEQ ID NO: 11 and/or the VL comprises or consists of the sequence SEQ ID NO: 12 with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids substituted by a different amino acid.

In one embodiment, the VH and/or the VL has/have an amino acid sequence that shares at least 70%, 75%, 80%, 85%, 90%, 95%, 96%, 97%, 98%, 99% or more of identity with SEQ ID NO: 11 and/or SEQ ID NO: 12, respectively.

An example of such an antibody is 21C8 antibody.

In one embodiment, the antibody or antigen-binding fragment thereof according to the present invention comprises:
a VH comprising or consisting of the sequence SEQ ID NO: 13; and
a VL comprising or consisting of the sequence SEQ ID NO: 14.

In one embodiment, the VH comprises or consists of the sequence SEQ ID NO: 13 and/or the VL comprises or consists of the sequence SEQ ID NO: 14 with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids substituted by a different amino acid.

In one embodiment, the VH and/or the VL has/have an amino acid sequence that shares at least 70%, 75%, 80%, 85%, 90%, 95%, 96%, 97%, 98%, 99% or more of identity with SEQ ID NO: 13 and/or SEQ ID NO: 14, respectively.

An example of such an antibody is 22B12 antibody.

As used herein, the phrase “characterized as having [ . . . ]amino acids being substituted by a different amino acid” in reference to a given sequence, refers to the occurrence, in said sequence, of conservative amino acid modifications.

As used herein, the expression “conservative amino acid modifications” refers to modifications that do not significantly affect or alter the binding characteristics of the antibody or antigen-binding fragment thereof containing the amino acid sequence. Such conservative modifications include amino acid substitutions, additions and deletions. Modifications can be introduced into an antibody or antigen-binding fragment thereof by standard techniques known in the art, such as site-directed mutagenesis and PCR-mediated mutagenesis.

Conservative amino acid substitutions are typically those in which an amino acid residue is replaced with an amino acid residue having a side chain with similar physicochemical properties. Specified variable region and CDR sequences may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acid insertions, deletions and/or substitutions. Where substitutions are made, preferred substitutions will be conservative modifications. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), β-branched side chains (e.g., threonine, valine, isoleucine) and aromatic side chains (e.g., tyrosine, phenylalanine, tryptophan, histidine). Thus, one or more amino acid residues within the CDRs and/or variable regions of the antibody or antigen-binding fragment thereof according to the present invention can be replaced with other amino acid residues from the same side chain family and the altered antibody can be tested for retained function (i.e., the properties set forth herein, such as, e.g., the binding to CD160-TM isoform) using the assays described herein. In another embodiments, a string of amino acids within the CDRs and/or variable regions of the antibody or antigen-binding fragment thereof according to the present invention can be replaced with a structurally similar string that differs in order and/or composition of side chain family members.

The invention also relates to a 21C8-like antibody i.e., to an antibody binding the same epitope as 21C8, or substantially the same epitope than 21C8. The present invention thus further relates to an antibody or antigen-binding fragment competing with 21C8 for binding to CD160-TM isoform.

The invention also relates to a 22B12-like antibody i.e., to an antibody binding the same epitope as 22B12, or substantially the same epitope than 22B12. The present invention thus further relates to an antibody or antigen-binding fragment competing with 22B12 for binding to CD160-TM isoform.

In one embodiment, the antibody or antigen-binding fragment thereof according to the present invention is polyclonal. In one embodiment, the antibody or antigen-binding fragment thereof according to the present invention is monoclonal.

In one embodiment, the antibody or antigen-binding fragment thereof according to the present invention is monovalent. In one embodiment, the antibody or antigen-binding fragment thereof according to the present invention is bivalent.

In one embodiment, the antibody or antigen-binding fragment thereof according to the present invention comprises a fully or substantially fully murine heavy chain constant region (abbreviated herein as C_{H}) and/or light chain constant region (abbreviated herein as C_{L}). In one embodiment, the constant region is of murine origin.

In one embodiment, the antibody or antigen-binding fragment thereof according to the present invention is a murine antibody or fragment thereof.

In one embodiment, the antibody or antigen-binding fragment thereof according to the present invention is a chimeric antibody or fragment thereof.

A “chimeric antibody or fragment thereof”, as used herein, refers to an antibody or antigen-binding fragment thereof comprising a first amino acid sequence linked to a second amino acid sequence with which it is not naturally linked in nature. The amino acid sequences may normally exist in separate proteins that are brought together in the fusion protein or may normally exist in the same protein but are placed in a new arrangement in the fusion protein. A chimeric protein may be created, for example, by chemical synthesis, or by creating and translating a polynucleotide in which the peptide regions are encoded in the desired relationship.

The term “chimeric antibody or fragment thereof” encompasses herein antibodies and antigen-binding fragments thereof in which
(a) the constant region, or a portion thereof, is altered, replaced or exchanged so that the variable region is linked to a constant region of a different or altered class, effector function and/or species, or an entirely different molecule which confers new properties to the chimeric antibody, e.g., an enzyme, toxin, hormone, growth factor, drug, etc.; or
(b) the variable region, or a portion thereof, is altered, replaced or exchanged with a variable region, or portion thereof, having a different or altered antigen specificity; or with corresponding sequences from another species or from another antibody class or subclass.

In one embodiment, the antibody or antigen-binding fragment thereof according to the present invention is a humanized antibody or fragment thereof.

A “humanized antibody or fragment thereof”, as used herein, refers to a chimeric antibody or antigen-binding fragment thereof which contains minimal sequence derived from a non-human immunoglobulin. It includes antibodies made by a non-human cell having variable and constant regions which have been altered to more closely resemble antibodies that would be made by a human cell, e.g., by altering the non-human antibody amino acid sequence to incorporate amino acids found in human germline immunoglobulin sequences. Humanized antibodies or antigen-binding fragments thereof according to the present invention may include amino acid residues not encoded by human germline immunoglobulin sequences (e.g., mutations introduced by random or site-specific mutagenesis in vitro or by somatic mutation in vivo), for example in the CDRs.

The term “humanized antibody or fragment thereof” also includes antibodies and antigen-binding fragments thereof in which CDR sequences derived from the germline of another mammalian species, such as a mouse, have been grafted onto human framework sequences. In other words, the term “humanized antibody or fragment thereof” refers to an antibody or antigen-binding fragment thereof in which the CDRs of a recipient human antibody are replaced by CDRs from a donor non-human antibody. Humanized antibodies or antigen-binding fragments thereof may also comprise residues of donor origin in the framework sequences. The humanized antibody or antigen-binding fragment thereof can also comprise at least a portion of a human immunoglobulin constant region. Humanized antibodies or antigen-binding fragments thereof may also comprise residues which are found neither in the recipient antibody nor in the imported CDR or framework sequences. Humanization can be performed using methods known in the art (e.g., Jones et al., 1986. Nature. 321(6069):522-5; Riechmann et al., 1988. Nature. 332(6162):323-7; Verhoeyen et al., 1988. Science. 239(4847):1534-6; Presta, 1992. Curr Opin Biotechnol. 3(4):394-8; U.S. Pat. No. 4,816,567), including techniques such as “superhumanizing” antibodies (e.g., Tan et al., 2002. J Immunol. 169(2):1119-25) and “resurfacing” (e.g., Staelens et al., 2006. Mol Immunol. 43(8):1243-57; Roguska et al., 1994. Proc Natl Acad Sci U S A. 91(3):969-73).

A “humanized antibody or fragment thereof” may retain a similar antigenic specificity as the original antibody or fragment. However, using certain methods of humanization, the affinity and/or specificity of binding of the antibody may be increased.

Methods for humanizing the antibody or antigen-binding fragment thereof according to the present invention are well-known in the art. The choice of human variable domains, both light and heavy, to be used in making the humanized antibody or antigen-binding fragment thereof is very important to reduce antigenicity. According to the so-called “best-fit” method, the sequence of the variable domain of an antibody or antigen-binding fragment thereof according to the present invention is screened against the entire library of known human variable-domain sequences. The human sequence that is closest to the mouse sequence is then accepted as the human framework (FR) for the humanized antibody or fragment (Sims et al., 1993. J Immunol. 151(4):2296-308; Chothia & Lesk, 1987. J Mol Biol. 196(4):901-17).

Another method for humanizing the antibody or antigen-binding fragment thereof according to the present invention uses a particular framework from the consensus sequence of all human antibodies of a particular subgroup of light or heavy chains. The same framework can be used for several different humanized antibodies or fragments (Carter et al., 1992. Proc Natl Acad Sci USA. 89(10):4285-9; Presta et al., 1993. J Immunol. 151(5):2623-32). It is further important that antibodies or fragments be humanized with retention of high affinity for CD160-TM isoform and other favorable biological properties. To achieve this goal, according to a preferred method, humanized antibodies and antigen-binding fragments thereof are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its epitope. In this way, CDR residues can be selected and combined from the consensus and import sequences so that the desired antibody characteristic, such as an increased affinity for CD160-TM isoform, is achieved. In general, the CDR residues are directly and most substantially involved in influencing antigen binding.

Another method for humanizing the antibody or antigen-binding fragment thereof according to the present invention is to use a transgenic or transchromosomic animal carrying parts of the human immune system for immunization. As a host, these animals have had their immunoglobulin genes replaced by functional human immunoglobulin genes. Thus, antibodies produced by these animals or in hybridomas made from the B cells of these animals are already humanized. Examples of such transgenic or transchromosomic animal include, without limitation:
the XenoMouse (Abgenix, Fremont, CA), described in U.S. Pat. Nos. 5,939,598, 6,075,181, 6,114,598, 6,150,584 and 6,162,963;
the HuMAb Mouse® (Medarex, Inc.), described in Lonberg et al., 1994. Nature. 368(6474):856-859; Lonberg & Huszar, 1995. Int Rev Immunol. 13(1):65-93; Harding & Lonberg, 1995. Ann N Y Acad Sci. 764:536-46; Taylor et al., 1992. Nucleic Acids Res. 20(23):6287-95; Chen et al., 1993. Int Immunol. 5(6):647-56; Tuaillon et al., 1993. Proc Natl Acad Sci USA. 90(8):3720-4; Choi et al., 1993. Nat Genet. 4(2):117-23; Chen et al., 1993. EMBO J. 12(3):821-30; Tuaillon et al., 1994. J Immunol. 152(6):2912-20; Taylor et al., 1994. Int Immunol. 6(4):579-91; Fishwild et al., 1996. Nat Biotechnol. 14(7):845-51;
the KM Mouse®, described in Patent application WO2002043478;
the TC mice, described in Tomizuka et al., 2000. Proc Natl Acad Sci USA. 97(2):722-7; and
the OmniRat™ (OMT, Inc.), described in Patent application WO2008151081; Geurts et al., 2009. Science. 325(5939):433; Menoret et al., 2010. Eur J Immunol. 40(10):2932-41.

Humanized antibodies and antigen-binding fragments thereof may also be produced according to various other techniques, such as by using, for immunization, other transgenic animals that have been engineered to express a human antibody repertoire (Jakobovitz et al, 1993. Nature. 362(6417):255-8), or by selection of antibody repertoires using phage display methods. Such techniques are known to the skilled person and can be implemented starting from monoclonal antibodies or antigen-binding fragments thereof as disclosed in the present application.

In one embodiment, the antibody or antigen-binding fragment thereof is from the IgG class.

In one embodiment, the antibody or antigen-binding fragment thereof is from the human IgG1 subclass. In one embodiment, the antibody or antigen-binding fragment thereof according to the present invention is thus an IgG1 antibody, preferably a human IgG1 antibody.

In another embodiment, the antibody or antigen-binding fragment thereof is from the human IgG2 subclass.

The Fc region of IgG antibodies interacts with cellular Fcγ receptors (FcγR) to stimulate and regulate downstream effector mechanisms. There are five activating receptors, namely FcγRI (CD64), FcγRIIa (CD32a), FcγRIIc (CD32c), FcγRIIIa (CD16a) and FcγRIIIb (CD16b), and one inhibitory receptor FcγRIIb (CD32b). The communication of IgG antibodies with the immune system is controlled and mediated by FcγRs, which relay the information sensed and gathered by antibodies to the immune system, providing a link between the innate and adaptive immune systems, and particularly in the context of biotherapeutics (Hayes J et al., 2016. J Inflamm Res 9: 209-219).

IgG subclasses vary in their ability to bind to FcγR and this differential binding determines their ability to elicit a range of functional responses. For example, in humans, FcγRIIIa is the major receptor involved in the activation of antibody-dependent cell-mediated cytotoxicity (ADCC) and IgG3 (followed closely by IgG1) display the highest affinities for this receptor, reflecting their ability to potently induce ADCC. IgG2 have been shown to have weak binding for this receptor.

Thus, the constant region of an antibody is important in the ability of an antibody to fix complement and mediate cell-dependent cytotoxicity and phagocytosis. Thus, as discussed herein, the isotype of an antibody may be selected on the basis of whether it is desirable for the antibody to mediate cytotoxicity/phagocytosis.

In one embodiment, the antibody or antigen-binding fragment thereof according to the present invention binds FcγR with high affinity, preferably binds an activating receptor with high affinity.

In one embodiment, the antibody or antigen-binding fragment thereof according to the present invention binds FcγRI and/or FcγRIIa and/or FcγRIIc and/or FcγRIIIa and/or FcγRIIIb with high affinity.

In one embodiment, the antibody or antigen-binding fragment thereof according to the present invention is an IgG1 antibody (preferably a human IgG1 antibody) or a fragment thereof, and binds to at least one Fc activating receptor. For example, the antibody or the antigen-binding fragment thereof may bind to one or more receptor selected from FcγRI, FcγRIIa, FcγRIIc, FcγRIIIa and FcγRIIIb. In one embodiment, the antibody or the antigen-binding fragment thereof is capable of binding to FcγRIIIa. In one embodiment, the antibody or the antigen-binding fragment thereof is capable of binding to FcγRIIa. In one embodiment, the antibody or the antigen-binding fragment thereof is capable of binding to FcγRIIIa, FcγRIIc and optionally FcγRI. In one embodiment, the antibody or the antigen-binding fragment thereof is capable of binding to FcγRIIIa, FcγRIIa and optionally FcγRI.

In one embodiment, the antibody or antigen-binding fragment thereof according to the present invention binds to at least one activating Fcγ receptor with a dissociation constant of less than about 10⁻⁶M, 10⁻⁷M, 10⁻⁸M, 10⁻⁹M or 10⁻¹⁰ M.

In one embodiment, the antibody or antigen-binding fragment thereof according to the present invention is an IgG1 antibody (preferably a human IgG1 antibody) or a fragment thereof and binds to FcγRI, FcγRIIa, FcγRIIc, FcγRIIIa, and/or FcγRIIIb with a higher affinity than it binds to FcγRIIb, with low affinity.

In one embodiment, the antibody or antigen-binding fragment thereof according to the present invention induces antibody dependent cellular cytotoxicity (ADCC). In one embodiment, the antibody or antigen-binding fragment thereof according to the present invention is from the IgG1 (preferably human IgG1) subclass and has ADCC activity.

The term “antibody-dependent cell-mediated cytotoxicity” or “ADCC” refers to a cell-mediated cytotoxicity induced in an antibody-dependent manner when the Fc region of said antibody bound to its antigen binds to the Fc receptor on effector cells such as natural killer cells, macrophages, neutrophils, eosinophils and mononuclear cells (e.g., peripheral blood mononuclear cells), thereby leading to lysis of the target cell. ADCC can be measured using assays that are known and available in the art (e.g., Clynes et al. (1998) Proc Natl Acad Sci USA 95, 652-6).

In one embodiment, the antibody or antigen-binding fragment thereof according to the present invention induces antibody-dependent cell-mediated phagocytosis (ADCP). In one embodiment, the antibody or antigen-binding fragment thereof according to the present invention is from the IgG1 (preferably human IgG1) subclass and has ADCP activity.

The term “antibody-dependent cell-mediated phagocytosis” (ADCP) or “opsonisation” refers to a cell-mediated reaction in which nonspecific cytotoxic cells (e.g., phagocytes, macrophages) that express Fc receptors (FcRs) recognize antibody bound on a target cell and induce phagocytosis of the target cell. ADCP can be measured using assays that are known and available in the art (e.g., Clynes et al. (1998) Proc Natl Acad Sci USA 95, 652-6).

In one embodiment, the antibody or antigen-binding fragment thereof according to the present invention induces complement-dependent cytotoxicity (CDC). In one embodiment, the antibody or antigen-binding fragment thereof according to the present invention is from the IgG1 (preferably human IgG1) subclass and has CDC activity.

The term “complement-dependent cytotoxicity” (CDC) refers to the induction of the lysis of antigen-expressing cells recognized by an antibody or antigen-binding fragment thereof of the invention in the presence of complement. The complement activation pathway is initiated by the binding of the first component of the complement system (C1q) to a molecule (e.g., an antibody) complexed with a cognate antigen. CDC can be measured using assays that are known and available in the art (e.g., Clynes et al. (1998) Proc Natl Acad Sci USA 95, 652-6; Gazzano-Santaro et al., J. Immunol. Methods, 202:163 (1996)).

In one embodiment, the antibody or antigen-binding fragment thereof according to the present invention comprises an Fc region that mediates ADCC, ADCP and/or CDC.

In one embodiment, the antibody or antigen-binding fragment thereof according to the present invention does not induce ADCC, ADCP and/or CDC.

In one embodiment, the antibody or antigen-binding fragment thereof according to the present invention does not comprise an Fc region that mediates ADCC, ADCP and/or CDC.

In one embodiment, the antibody or antigen-binding fragment thereof according to the present invention lacks an Fc domain (e.g., lacks a C_{H}2 and/or C_{H}3 domain) or comprises an Fc domain of IgG2 or IgG4 isotype (preferably of human IgG2 or IgG4).

The antibodies or antigen-binding fragments thereof according to the present invention may be produced by any technique known in the art, such as, without limitation, any chemical, biological, genetic or enzymatic technique, either alone or in combination.

Thus, another object of the present invention is a method of producing and purifying the antibody or an antigen-binding fragment thereof as described herein.

In one embodiment, the method comprises:
introducing in vitro or ex vivo a recombinant nucleic acid or a vector as described herein into a competent host cell;
culturing in vitro or ex vivo the host cells transformed with the nucleic acid or expression vector, under conditions suitable for expression of the antibody or antigen-binding fragment thereof,
optionally, selecting the cells which express and/or secrete said antibody or fragment thereof, and
recovering the expressed antibody or antigen-binding fragment thereof.

This recombinant process can be used for large scale production of antibodies or antigen-binding fragments thereof, including monoclonal antibodies intended for in vitro, ex vivo and/or in vivo therapeutic uses.

In one embodiment, the expressed antibody or antigen-binding fragment thereof is further purified.

Methods to purify an antibody or antigen-binding fragment thereof, are well-known in the art and include, without limitation, protein A-Sepharose, gel electrophoresis, chromatography, preferably by affinity chromatography, more preferably by affinity chromatography on protein L agarose.

It will also be appreciated that the antibodies or antigen-binding fragments thereof according to the present invention can be modified using known methods in the art.

In one embodiment, the antibody or antigen-binding fragment thereof according to the present invention is an engineered antibody or fragment thereof.

Engineered antibodies or fragment thereof according to the present invention include those in which modifications have been made to framework residues within VH and/or VL, e.g., to improve the properties of the antibody. Typically, such framework modifications are made to decrease the immunogenicity of the antibody. For example, one approach is to “back-mutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “back-mutated” to the germline sequence by, for example, site-directed mutagenesis or PCR-mediated mutagenesis. Such “back-mutated” antibodies are also intended to be encompassed by the invention. Another type of framework modification involves mutating one or more residues within the framework region, or even within one or more CDR regions, to remove T cell-epitopes to thereby reduce the potential immunogenicity of the antibody. This approach is also referred to as “deimmunization” and is described in further detail in U.S. Patent Publication No. 20030153043 by Carr et al.

In one embodiment, the antibody or antigen-binding fragment thereof according to the present invention is engineered to elicit an enhanced, increased or improved ADCC, ADCP, and/or CDC response.

Methods to increase ADCC, ADCP and/or CDC are well known in the art. For example, ADCC may be increased by methods that eliminate the fucose moiety from the antibody glycan, such as by production of the antibody in a YB2/0 cell line, or though the introduction of specific mutations on the Fc portion of human IgG1 (e.g., S298A/E333A/K334A, S239D/I332E/A330L,G236A/S239D/A330L/I332E) (Lazar et al. (2006) Proc Natl Acad Sci USA 103, 2005-2010; Smith et al. (2012) Proc Natl 25 Acad Sci USA 109, 6181-6). ADCP may also be increased by the introduction of specific mutations on the Fc portion of human IgG1 (Richards et al. (2008) Mol Cancer Ther 7, 2517-27). CDC response may be increased with mutations in the antibody that increase the affinity of C1q binding (Idusogie et al. (2001) J Immunol 166, 2571-5).

In one embodiment, the antibody or antigen-binding fragment thereof according to the present invention is engineered to elicit a decreased ADCC, ADCP, and/or CDC response.

Methods to decrease or abolish ADCC, ADCP and/or CDC are also well known in the art. For example, ADCC may be decreased or abolished by methods modifying the glycosylation profile of the Fc domain of the immunoglobulin. CDC can be decreased or abolished by the replacement of one or more amino acids by other amino acid such that the antibody has altered C2q binding (U.S. Pat. No. 6,194,551 by Idusogie et al.)

As used herein, the terms “enhanced, increased or improved ADCC, ADCP, and/or CDC response” and “decreased ADCC, ADCP, and/or CDC response” are relative to the ADCC, ADCP, and/or CDC response induced by the modified antibody or fragment thereof according to the present invention as compared to the ADCC, ADCP, and/or CDC response induced with other anti-CD160-TM antibodies, for example unmodified anti-CD160-TM monoclonal antibodies.

In one embodiment, the antibody or antigen-binding fragment thereof according to the present invention is engineered to modify its glycosylation.

For example, the antibody or fragment thereof according to the invention is aglycosyled (i.e., the antibody or fragment thereof lacks glycosylation). Glycosylation can be altered to, for example, increase the affinity of the antibody for the antigen or alter the ADCC activity of the antibody. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Additionally or alternatively, an antibody can be made that has an altered type of glycosylation, such as a hypofucosylated or non-fucosylated antibody having reduced amounts of or no fucosyl residues or an antibody having increased bisecting GlcNac structures. Such altered fucosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the present invention to thereby produce an antibody with altered glycosylation. Alternatively, the antibody (preferably the monoclonal antibody) of the present invention can be produced in yeasts or filamentous fungi engineered for mammalian-like glycosylation pattern and capable of producing antibodies lacking fucose as glycosylation pattern.

In one embodiment, the antibody or antigen-binding fragment thereof according to the present invention is a pegylated antibody or fragment thereof.

An antibody or fragment thereof can be pegylated to, for example, increase the biological (e.g., serum) half-life of the antibody. To pegylate an antibody or a fragment thereof, the antibody or fragment thereof typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (DY12-DY120) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies or fragments according to the present invention, such as, for example, as described in EP0154316 and EP0401384 (incorporated herein by reference).

The present invention further relates to a fusion protein comprising an antibody or an antigen-binding fragment as described hereinabove.

The present invention further relates to an antibody or an antigen-binding fragment as described hereinabove being conjugated.

In one embodiment, the antibody or the antigen-binding fragment according to the present invention is conjugated to a therapeutic moiety, i.e., a drug.

The therapeutic moiety can be, e.g., a cytotoxin, a chemotherapeutic agent, a cytokine, an immunosuppressant, an immune stimulator, a lytic peptide, or a radioisotope. Such conjugates are referred to herein as an “antibody-drug conjugates” or “ADCs”.

Examples of radioisotopes include, but are not limited to, ⁹⁰Y ¹³¹I, or ⁶⁷Cu.

In one embodiment, the antibody or the antigen-binding fragment according to the present invention is conjugated to a cytotoxic moiety.

The cytotoxic moiety may, for example, be selected from the group consisting of taxol; cytochalasin B; gramicidin D; ethidium bromide; emetine; mitomycin; etoposide; tenoposide; vincristine; vinblastine; colchicin; doxorubicin; daunorubicin; dihydroxy anthracin dione; a tubulin-inhibitor such as maytansine or an analog or derivative thereof; an antimitotic agent such as monomethyl auristatin E or F or an analog or derivative thereof; dolastatin 10 or 15 or an analogue thereof; irinotecan or an analogue thereof, mitoxantrone; mithramycin; actinomycin D; 1-dehydrotestosterone; a glucocorticoid; procaine; tetracaine; lidocaine; propranolol; puromycin; calicheamicin or an analog or derivative thereof; an antimetabolite such as methotrexate, 6 mercaptopurine, 6 thioguanine, cytarabine, fludarabin, 5 fluorouracil, decarbazine, hydroxyurea, asparaginase, gemcitabine, or cladribine; an alkylating agent such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C; a platinum derivative such as cisplatin or carboplatin; duocarmycin A, duocarmycin SA, rachelmycin (CC-1065); an antibiotic such as dactinomycin, bleomycin, daunorubicin, doxorubicin, idarubicin, mithramycin, mitomycin, mitoxantrone, plicamycin, anthramycin (AMC)); pyrrolo[2,1-c][1,4]-benzodiazepines (PDB); diphtheria toxin and related molecules such as diphtheria A chain and active fragments thereof and hybrid molecules, ricin toxin such as ricin A or a deglycosylated ricin A chain toxin, cholera toxin, a Shiga-like toxin such as SLT I, SLT II, SLT IIV, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins such as PAPI, PAPII, and PAP-S, Momordica charantia inhibitor, curcin, crotin, Sapaonaria officinalis inhibitor, gelonin, mitogellin, restrictocin, phenomycin, and enomycin toxins; ribonuclease (RNase); DNase I, Staphylococcal enterotoxin A; pokeweed antiviral protein; diphtherin toxin; and Pseudomonas endotoxin.

In one embodiment, the antibody or the antigen-binding fragment according to the present invention is conjugated to a cell-killing cytotoxic drug (payload) selected from the group comprising or consisting of ozogamicin, vedotin, emtansine, deruxtecan, govitecan, mafodotin, pasudotox and tesirine.

In one embodiment, the antibody or the antigen-binding fragment according to the present invention is conjugated to a detectable label.

Labels for the anti-CD160-TM antibody or antigen-binding fragment thereof according to the present invention that may be used include, without limitation, various enzymes, prosthetic groups, fluorescent materials, luminescent materials, magnetic agents and radioactive materials.

Examples of such enzymes include, but are not limited to, horseradish peroxidase, alkaline phosphatase, beta-galactosidase or acetylcholinesterase.

Examples of prosthetic group complexes include, but are not limited to, streptavidin/biotin and avidin/biotin.

Examples of fluorescent materials include, but are not limited to, organic molecules, such as fluorescent proteins or fluorescent small organic molecules, and inorganic molecules. Examples of fluorescent materials include, without limitation, umbelliferone, fluorescein, fluorescein isothiocyanate, rhodamine, dichlorotriazinylamine fluorescein, dansyne chloride, phycoerythrin, green fluorescent protein (GFP) and its derivatives or quantum dots.

Examples of luminescent material include but are not limited to, luminal.

Examples of magnetic agents include gadolinium; and examples of suitable radioactive material include ¹²⁵I, ¹³¹I, ³⁵S or ³H.

Techniques for conjugating molecule to antibodies or antigen-binding fragments thereof are well-known in the art. Typically, the nucleic acid molecule is covalently attached to lysines or cysteines on the antibody or fragment thereof, through N-hydroxysuccinimide ester or maleimide functionality respectively. Methods of conjugation using engineered cysteines or incorporation of unnatural amino acids have been reported to improve the homogeneity of the conjugate.

The present invention further relates to an antibody or an antigen-binding fragments as described hereinabove being bispecific.

The present invention further relates to an antibody or an antigen-binding fragment as described hereinabove that is not conjugated.

Another object of the invention is an isolated nucleic acid encoding an antibody or an antigen-binding fragment thereof, or a fusion protein as described hereinabove.

An “isolated nucleic acid”, as used herein, is intended to refer to a nucleic acid that is substantially separated from other genome DNA sequences as well as proteins or complexes such as ribosomes and polymerases, which naturally accompany a native sequence. The term embraces a nucleic acid sequence that has been removed from its naturally occurring environment, and includes recombinant or cloned DNA isolates and chemically synthesized analogues or analogues biologically synthesized by heterologous systems. A substantially pure nucleic acid includes isolated forms of the nucleic acid. Of course, this refers to the nucleic acid as originally isolated and does not exclude genes or sequences later added to the isolated nucleic acid by the hand of man.

In one embodiment, the isolated nucleic acid is purified.

In one embodiment, the isolated nucleic acid is purified to:
(1) greater than 80%, 85%, 90%, 91%, 92%, 93%, 94%, 95% or more by weight of nucleic acid as determined by absorbance methods or fluorescence methods (such as, e.g., by measuring the ratio of absorbance at 260 and 280 nm (A_{260/280})), and most preferably greater than 96%, 97%, 98% or 99% by weight; or
(2) homogeneity as shown by agarose gel electrophoresis and using an intercalating agent such as ethidium bromide, SYBR Green, GelGreen or the like.

In one embodiment, the nucleic acid encodes at least a heavy chain variable region or a light chain variable region of the antibody or antigen-binding fragment thereof according to the present invention.

In one embodiment, the nucleic acid encodes variable and constant regions of the antibody or antigen-binding fragment thereof according to the present invention.

In one embodiment, the nucleic acid may encode heavy and light chains of the antibody or antigen-binding fragment thereof on separate nucleic acids or on the same nucleic acid molecule.

In one embodiment, the nucleic acid comprises or consists of a sequence encoding the VH of the antibody or antigen-binding fragment thereof according to the invention.

In one embodiment, the nucleic acid comprises or consists of a sequence encoding the VH of the antibody or antigen-binding fragment thereof according to the invention, wherein said sequence is selected from the group comprising or consisting of SEQ ID NO: 17, SEQ ID NO: 19 and any sequence sharing at least 70%, 75%, 80%, 85%, 90%, 95%, 96%, 97%, 98%, 99% or more identity with SEQ ID NOs: 17 and 19.

SEQ ID NO: 17
CAGGTCCAACTGCAGCAACCTGGGTCTGAGCTGGTGAGGCCTGGAGCTT
CAGTGAGGCTGTCCTGCAAGGCTTCTGGCTACACATTCACCAGCTACTG
GATGCACTGGGTGAGGCAGAGGCATGGACAAGGCCTTGAGTGGATTGGA
AATATTTATCCTGGTAGTGGTTTTACTAATTACGATGAGAAATTCAAGA
ACAAGGGCACACTGACTGTAGACACATCCTCCAGTACAGCCTACATGCA
CCTCAGCAGCCTGACATCTGAGGACTCTGCGGTCTATTACTGTTCATTG
GACGAAGGGTTTGCTTACTGGGGCCAAGGGACTCTGGTCACTGTCTCTG
CA
SEQ ID NO: 19
CAGGTGCAGCTGAAGCAGTCAGGACCTGGCCTAGTGCAGCCCTCACAGA
GCCTGTCCATAATGTGCACAGTCTCTGGTTTCTCATTAACTAGCTATGG
TTTACACTGGGTTCGCCAGTCTCCAGGAAAGGGTCTGGAGTGGCTGGGA
GTGATATGGAGAGGTGGAAACACAGACTACAATGCAGATTTCATGTCCA
GACTGACCATCACCAAGGACAATTCCAAGAGCCAAGTTTTCTTTAAAAT
GAACAGTCTGCAAGCTGATGACACTGCCGTTTACTACTGTGTCAAAAAT
GGCGGTAACTCCAAGGACTATGCTTTGGACTACTGGGGTCAAGGAACCT
CAGTCACCGTCTCCTCA

In one embodiment, the nucleic acid comprises or consists of a sequence encoding the VL of the antibody or antigen-binding fragment thereof according to the invention.

In one embodiment, the nucleic acid comprises or consists of a sequence encoding the VL of the antibody or antigen-binding fragment thereof according to the invention, wherein said sequence is selected from the group comprising or consisting of SEQ ID NO: 18, SEQ ID NO: 20 and any sequence sharing at least 70%, 75%, 80%, 85%, 90%, 95%, 96%, 97%, 98%, 99% or more identity with SEQ ID NOs: 18 and 20.

SEQ ID NO: 18
GACATCCAAATGAACCAGTCTCCATCCAGTCTGTCTGCATCCCTCGGAG
ACACAATTACCATCACTTGCCGTGCCAGTCAGGGCATTAATATTTGGTT
AAACTGGTACCAGCAGAAACCAGGAAATATTCCTAAACGATTGATCTAT
AAGGCTTCCAACTTGCACACAGGAGTCCCACCAAGGTTTAGAGGCAGTG
GATCTGGAACAGATTTCACATTAACTATCAGCAGCCTACAGCCTGAAGA
CATTGCCACTTACTACTGTCTACAGAGTCAAAGTTATCCATTCACGTTC
GGCTCGGGGACAAAGTTGGAAATTAGA
SEQ ID NO: 20
AACATTGTAATGACCCAATCTCCCAAATCCATGTCCATGTCAGTAGGAG
AGAGGGTCACCTTGAGCTGCAAGGCCAGTGAGAATGTGGGTATTTATGT
ATCCTGGTATCAACAGAAACCAGAGCAGTCTCCTAAACTGCTGATATAC
GGGTCATCCAACCGGTACACTGGGGTCCCTGATCGCTTCACAGGCAGTG
GATCTGCAACAGATTTCACTCTGATCATCAGCAGTGTTCAGGCTGAAGA
CCTTGCAGATTATCACTGTGGACAGAGTTACAGTTATCCGTACACGTTC
GGAGGGGGGACCAAGCTGGAAATAAAA

In one embodiment, the nucleic acid according to the present invention comprises or consists of:
a sequence encoding the VH of the antibody or antigen-binding fragment thereof according to the present invention; and
a sequence encoding the VL of the antibody or antigen-binding fragment thereof according to the present invention.

In one embodiment, the nucleic acid according to the present invention comprises or consists of:
a sequence encoding the VH comprising or consisting of the sequence SEQ ID NO: 17; and
a sequence encoding the VL comprising or consisting of the sequence SEQ ID NO: 18.

In one embodiment, said nucleic acid encodes the VH and the VL of the 21C8 antibody.

In one embodiment, the nucleic acid according to the present invention comprises or consists of:
a sequence encoding the VH comprising or consisting of the sequence SEQ ID NO: 19; and
a sequence encoding the VL comprising or consisting of the sequence SEQ ID NO: 20.

In one embodiment, said nucleic acid encodes the VH and the VL of the 22B12 antibody.

In one embodiment, the nucleic acid according to the present invention comprises a sequence encoding a fully or substantially fully human C_{H} and/or C_{L} of the antibody or antigen-binding fragment thereof according to the present invention. In such embodiment, constant regions may be derived from any human antibody constant regions.

In one embodiment, the nucleic acid according to the present invention comprises a sequence encoding a fully or substantially fully murine C_{H} and/or C_{L} of the antibody or antigen-binding fragment thereof according to the invention. In such embodiment, constant regions may be derived from any murine antibody constant regions.

In one embodiment, the nucleic acid according to the present invention comprises or consists of a sequence encoding the heavy chain of the chimeric antibody or antigen-binding fragment thereof according to the invention. In one embodiment, the nucleic acid according to the present invention comprises or consists of a sequence encoding the light chain of the chimeric antibody or antigen-binding fragment thereof according to the invention.

In one embodiment, the nucleic acid according to the present invention comprises or consists of a sequence encoding the heavy chain of the humanized antibody or antigen-binding fragment thereof according to the invention. In one embodiment, the nucleic acid according to the present invention comprises or consists of a sequence encoding the light chain of the humanized antibody or antigen-binding fragment thereof according to the invention.

Typically, said nucleic acid is a DNA or RNA molecule, which may be included in any suitable vector, such as for example plasmid, cosmid, episome, artificial chromosome, phage or a viral vector.

Thus, another object of the present invention is an expression vector comprising a nucleic acid as described hereinabove.

The terms “vector”, “cloning vector” and “expression vector” mean the vehicle by which a DNA or RNA sequence (e.g., a foreign gene) can be introduced into a host cell, so as to transform a host and promote expression (e.g. transcription and translation) of the introduced sequence. Such vectors may comprise regulatory elements, such as a promoter, enhancer, terminator and the like, to cause or direct expression of said protein or antibody or antigen-binding fragment thereof upon administration to a host. Examples of promoters and enhancers used in the expression vector for animal cell include early promoter and enhancer of SV40, LTR promoter and enhancer of Moloney mouse leukemia virus, promoter and enhancer of immunoglobulin H chain and the like. Any expression vector for animal cell can be used, so long as a gene encoding the protein, antibody or fragment thereof can be inserted and expressed. Examples of suitable vectors include pAGE107, pAGE103, pHSG274, pKCR, pSG1 beta d2-4 and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found in the art.

In one embodiment, the expression vector according to the present invention comprises a sequence encoding the VH of the antibody or antigen-binding fragment thereof according to the invention, operably linked to regulatory elements.

In one embodiment, the expression vector according to the present invention comprises a sequence selected from the group comprising or consisting of SEQ ID NO: 17, SEQ ID NO: 19 and any sequence sharing at least 70%, 75%, 80%, 85%, 90%, 95%, 96%, 97%, 98%, 99% or more identity with SEQ ID NO: 17 and 19, operably linked to regulatory elements.

In one embodiment, the expression vector according to the present invention comprises a sequence encoding the VL of the antibody or antigen-binding fragment thereof according to the invention, operably linked to regulatory elements.

In one embodiment, the expression vector according to the present invention comprises a sequence selected from the group comprising or consisting of SEQ ID NO: 18, SEQ ID NO: 20, and any sequence sharing at least 70%, 75%, 80%, 85%, 90%, 95%, 96%, 97%, 98%, 99% or more identity with SEQ ID NO: 18 and 20, operably linked to regulatory elements.

In one embodiment, the expression vector according to the present invention comprises:
a sequence encoding the VH of the antibody or antigen-binding fragment thereof according to the invention, operably linked to regulatory elements; and
a sequence encoding the VL of the antibody or antigen-binding fragment thereof according to the invention, operably linked to regulatory elements.

In one embodiment, the expression vector according to the present invention comprises:
a sequence encoding the VH comprising or consisting of the sequence SEQ ID NO: 17 or any sequence sharing at least 70%, 75%, 80%, 85%, 90%, 95%, 96%, 97%, 98%, 99% or more identity with SEQ ID NO: 17, operably linked to regulatory elements, and
a sequence encoding the VL comprising or consisting of the sequence SEQ ID NO: 18 or any sequence sharing at least 70%, 75%, 80%, 85%, 90%, 95%, 96%, 97%, 98%, 99% or more identity with SEQ ID NO: 18, operably linked to regulatory elements.

In one embodiment, the expression vector according to the present invention comprises:
a sequence encoding the VH comprising or consisting of the sequence SEQ ID NO: 19 or any sequence sharing at least 70%, 75%, 80%, 85%, 90%, 95%, 96%, 97%, 98%, 99% or more identity with SEQ ID NO: 19, operably linked to regulatory elements, and
a sequence encoding the VL comprising or consisting of the sequence SEQ ID NO: 20 or any sequence sharing at least 70%, 75%, 80%, 85%, 90%, 95%, 96%, 97%, 98%, 99% or more identity with SEQ ID NO: 20, operably linked to regulatory elements.

In one embodiment, the expression vector according to the present invention comprises a sequence encoding the C_{H} of the antibody or antigen-binding fragment thereof according to the invention, operably linked to regulatory elements, wherein said C_{H} may be derived from any human antibody C_{H}.

In one embodiment, the expression vector according to the present invention comprises a sequence encoding the C_{L} of the antibody or antigen-binding fragment thereof according to the invention, operably linked to regulatory elements, wherein said C_{L} may be derived from any human antibody C_{L}.

In one embodiment, the expression vector according to the present invention comprises a sequence encoding the C_{H} of the antibody or antigen-binding fragment thereof according to the invention, operably linked to regulatory elements, wherein said C_{H} may be derived from any murine antibody C_{H}.

In one embodiment, the expression vector according to the present invention comprises a sequence encoding the C_{L} of the antibody or antigen-binding fragment thereof according to the invention, operably linked to regulatory elements, wherein said C_{L} may be derived from any murine antibody C_{L}.

In one embodiment, the expression vector according to the present invention comprises a sequence encoding the heavy chain of the chimeric antibody or antigen-binding fragment thereof according to the present invention, operably linked to regulatory elements.

In one embodiment, the expression vector according to the present invention comprises a sequence encoding the light chain of the chimeric antibody or antigen-binding fragment thereof according to the present invention, operably linked to regulatory elements.

In one embodiment, the expression vector according to the present invention comprises a sequence encoding the heavy chain of the humanized antibody or antigen-binding fragment thereof according to the present invention, operably linked to regulatory elements.

In one embodiment, the expression vector according to the present invention comprises a sequence encoding the light chain of the humanized antibody or antigen-binding fragment thereof according to the present invention, operably linked to regulatory elements.

In one embodiment, the expression vector according to the present invention is monocistronic.

By “monocistronic”, it is meant that a single nucleic acid is expressed in a single expression vector.

In one embodiment, the expression vector according to the present invention is polycistronic.

By “polycistronic”, it is meant that at least two or more nucleic acids are expressed in a single expression vector.

Another object of the invention is an isolated host cell comprising the expression vector as described hereinabove.

Said host cell may be used for the recombinant production of the antibodies or antigen-binding fragments thereof according to the present invention.

In an embodiment, host cells may be prokaryote, yeast, or eukaryote cells, preferably mammalian cells, such as, for example: monkey kidney CV1 line transformed by SV40 (COS-7, ATCC CRL 1651); human embryonic kidney line (293 or 293 cells subcloned for growth in suspension culture, Graham et al., J. Gen. Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse Sertoli cells (TM4, Mather, Biol. Reprod. 23:243-251 (1980)); mouse myeloma cells SP2/0-AG14 (ATCC CRL 1581; ATCC CRL 8287) or NSO (HPA culture collections no. 85110503); monkey kidney cells (CVl ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2), as well as DSM's PERC-6 cell line. Expression vectors suitable for use in each of these host cells are also generally known in the art. It should be noted that the term “host cell” generally refers to a cultured cell line. In one embodiment, whole human beings into which an expression vector encoding an antibody or an antigen-binding fragment thereof according to the present invention has been introduced are excluded from the definition of a “host cell”.

Another object of the present invention is a composition comprising, consisting essentially of or consisting of an antibody or antigen-binding fragment, a nucleic acid, an expression vector or a fusion protein as described hereinabove.

Another object of the present invention is a pharmaceutical composition comprising, consisting essentially of or consisting of an antibody or antigen-binding fragment, a nucleic acid, an expression vector or a fusion protein as described hereinabove, and at least one pharmaceutically acceptable excipient.

As used herein, “consisting essentially of”, with reference to a composition, means that the antibody or antigen-binding fragment thereof, nucleic acid, expression vector or fusion protein is the only one therapeutic agent or agent with a biologic activity within said composition.

The term “pharmaceutically acceptable excipient” includes any and all solvents, dispersion media, coatings, antibacterial and antifungal agents, isotonic and absorption delaying agents and the like. Said excipient does not produce an adverse, allergic or other untoward reaction when administered to an animal, preferably a mammal, more preferably a human. For human administration, preparations should meet sterility, pyrogenicity, and general safety and purity standards as required by regulatory offices, such as, for example, FDA Office or EMA.

Examples of pharmaceutically acceptable excipients that may be used in the compositions of the present invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

In one embodiment, the pharmaceutical compositions according to the present invention comprise vehicles which are pharmaceutically acceptable for a formulation capable of being injected to a subject. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

Another object of the present invention is a medicament comprising, consisting essentially of or consisting of an antibody or antigen-binding fragment, a nucleic acid, an expression vector or a fusion protein as described hereinabove.

In one embodiment, the composition, pharmaceutical composition or medicament according to the present invention is to be formulated for administration to the subject.

In one embodiment, the composition, pharmaceutical composition or medicament according to the present invention is to be administered parenterally, by inhalation spray, rectally, nasally, or via an implanted reservoir.

In one embodiment, the composition, pharmaceutical composition or medicament is to be administered by injection, including, without limitation, subcutaneous, intravenous, intramuscular, intra-articular, intra-synovial, intrasternal, intrathecal, intrahepatic, intralesional and intracranial injection or infusion techniques.

Examples of forms adapted for injection include, but are not limited to, solutions, such as, for example, sterile aqueous solutions, gels, dispersions, emulsions, suspensions, solid forms suitable for using to prepare solutions or suspensions upon the addition of a liquid prior to use, such as, for example, powder, liposomal forms and the like.

Sterile injectable forms of the compositions, pharmaceutical compositions or medicaments of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In one embodiment, the antibody or antigen-binding fragment thereof, the fusion protein, the nucleic acid, the expression vector, the composition, the pharmaceutical composition or the medicament according to the present invention is to be administered to the subject in need thereof in a therapeutically effective amount.

It will be however understood that the total daily usage of the antibody or antigen-binding fragment thereof, the fusion protein, the nucleic acid, the expression vector, the composition, the pharmaceutical composition or the medicament according to the present invention will be decided by the attending physician within the scope of sound medical judgment. The specific therapeutically effective dose level for any particular patient will depend upon a variety of factors including the disease being treated and the severity of the disease; activity of the antibody or antigen-binding fragment thereof, fusion protein, nucleic acid, or expression vector employed; the age, body weight, general health, sex and diet of the subject; the time of administration, route of administration, and rate of excretion of the specific antibody or antigen-binding fragment thereof, fusion protein, nucleic acid, or expression vector employed; the duration of the treatment; drugs used in combination or coincidental with the specific antibody or antigen-binding fragment thereof, fusion protein, nucleic acid, or expression vector employed; and like factors well known in the medical arts. For example, it is well within the skill of the art to start doses of the compound at levels lower than those required to achieve the desired therapeutic effect and to gradually increase the dosage until the desired effect is achieved. The total dose required for each treatment may be administered by multiple doses or in a single dose.

The daily dosage of the antibodies or antigen-binding fragments thereof according to the preset invention may be varied over a wide range from 10 to 1000 mg per adult per day.

The present invention further relates to an antibody or antigen-binding fragment, a nucleic acid, an expression vector or a fusion protein as described hereinabove, for use as a medicament, i.e., for use in treating a disease, disorder or condition in a subject in need thereof.

The present invention further relates to a composition, a pharmaceutical composition or a medicament as described hereinabove, for use as a medicament, i.e., for use in treating a disease, disorder or condition in a subject in need thereof.

The present invention further relates to a method for treating a disease, disorder or condition in a subject in need thereof, comprising administering to the subject an antibody or antigen-binding fragment, a nucleic acid, an expression vector or a fusion protein as described hereinabove.

The present invention further relates to a method for treating a disease, disorder or condition in a subject in need thereof, comprising administering to the subject the composition, pharmaceutical composition or medicament as described hereinabove.

The present invention further relates to the use of an antibody or antigen-binding fragment, a nucleic acid, an expression vector or a fusion protein as described hereinabove for the manufacture of a medicament for treating a disease, disorder or condition in a subject in need thereof.

Diseases, disorders or conditions that can be treated in the present invention include, without limitation, cancers, infectious diseases, inflammatory diseases and/or autoimmune diseases. Another example of diseases, disorders or conditions that can be treated in the present invention is paroxysmal nocturnal hemoglobinuria.

As used herein, the term “cancer” has its general meaning in the art and includes, but is not limited to, solid tumors and blood borne tumors. The term cancer includes, without limitation, diseases of the skin, tissues, organs, bone, cartilage, blood and vessels. The term “cancer” further encompasses both primary and metastatic cancers.

Examples of cancers that can be treated in the present invention include, but are not limited to, cancer cells from the bladder, blood, bone, bone marrow, brain, breast, colon, esophagus, gastrointestinal, gum, head, kidney, liver, lung, nasopharynx, neck, ovary, prostate, skin, stomach, testis, tongue, endometrial, pancreas or uterus.

In addition, the cancer may be selected in the following non-limiting list: malignant neoplasm; undifferentiated carcinoma; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; malignant gastrinoma; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma associated with familial polyposis coli; solid carcinoma; malignant carcinoid tumor; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non-encapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease of the breast; acinar cell carcinoma; adenosquamous carcinoma; adenocarcinoma with squamous metaplasia; malignant thymoma; malignant ovarian stromal tumor; malignant thecoma; malignant granulosa cell tumor; malignant roblastoma; Sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malign melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; malignant lymphoma—small lymphocytic; malignant diffuse large cell lymphoma; malignant follicular lymphoma; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In one embodiment, the cancer is a breast cancer. Examples of subtypes of breast cancers that may be treated by the antibody or antigen-binding fragment of the present invention include, without limitation, luminal A breast cancer, luminal B breast cancer, triple-negative breast cancer (TNBC), HER2-positive breast cancer.

As used herein, luminal A breast cancer is positive for estrogen receptor and/or progesterone receptor, negative for HER2, and has low levels of the protein Ki-67 (also known as MKI67 or antigen KI-67). As used herein, luminal B breast cancer is positive for estrogen receptor and/or progesterone receptor, positive for HER2, and has high levels of Ki-67. As used herein, TNBC is negative for estrogen receptor and progesterone receptor, and negative for HER2. As used herein, HER2-positive breast cancer is negative for estrogen receptor and progesterone receptor, and positive for HER2.

In one embodiment, the cancer is a breast cancer selected from the group comprising or consisting of luminal A breast cancer, luminal B breast cancer, triple-negative breast cancer (TNBC) and HER2-positive breast cancer.

In one embodiment, the cancer is luminal A breast cancer. In one embodiment, the cancer is luminal B breast cancer. In one embodiment, the cancer is TNBC. In one embodiment, the cancer is HER2-positive breast cancer.

As used herein, the term “infectious disease” includes any infection caused by viruses, bacteria, protozoa, molds or fungi.

In some embodiments, the viral infection comprises infection by one or more viruses selected from the group comprising, but not limited to, Arenaviridae, Astroviridae, Birnaviridae, Bromoviridae, Bunyaviridae, Caliciviridae, Closteroviridae, Comoviridae, Cystoviridae, Flaviviridae, Flexiviridae, Hepevirus, Leviviridae, Luteoviridae, Mononegavirales, Mosaic Viruses, Nidovirales, Nodaviridae, Orthomyxoviridae, Picobirnavirus, Picornaviridae, Potyviridae, Reoviridae, Retroviridae, Sequiviridae, Tenuivirus, Togaviridae, Tombusviridae, Totiviridae, Tymoviridae, Hepadnaviridae, Herpesviridae, Paramyxoviridae or Papillomaviridae viruses. Relevant taxonomic families of RNA viruses include, without limitation, Astroviridae, Birnaviridae, Bromoviridae, Caliciviridae, Closteroviridae, Comoviridae, Cystoviridae, Flaviviridae, Flexiviridae, Hepevirus, Leviviridae, Luteoviridae, Mononegavirales, Mosaic Viruses, Nidovirales, Nodaviridae, Orthomyxoviridae, Picobirnavirus, Picornaviridae, Potyviridae, Reoviridae, Retroviridae, Sequiviridae, Tenuivirus, Togaviridae, Tombusviridae, Totiviridae, and Tymoviridae viruses.

In some embodiments, the viral infection comprises infection by one or more viruses selected from the group comprising, but not limited to, adenovirus, Alfuy virus, Banzi virus, bovine diarrhea virus, coronavirus, Coxsackie virus, Crimean-Congo virus, Dengue virus, Ebola virus, encephalitis viruses (including Japanese Encephalitis virus, California Encephalitis virus, Venezuelan equine encephalitis virus, Western equine encephalitis virus, Eastern equine encephalitis virus, St. Louis encephalitis virus, tick-borne encephalitis virus), guanarito virus, hantavirus, hepatitis virus, Ilheus virus, immunodeficiency virus, influenza viruses including influenza A and influenza B viruses (including human, avian, and swine) and parainfluenza virus, junin virus, Kokobera virus, Kunjin virus, Kyasanur Forest disease virus, La Crosse virus, Lassa virus, louping-ill virus, lymphocytic choriomeningitis virus, measles virus, machupo virus, Marburg virus, Murray Valley virus, pachindae viruses, Pichinde virus, poliovirus, Powassan virus, Punta Toro virus, respiratory syncytial virus, rhinovirus, Rift Valley Fever virus, Rocio virus, severe acute respiratory syndrome (SARS), small pox virus, Tacaribe virus, West Nile and yellow fever viruses.

Examples of bacterial infections that may be treated in the present invention include, but are not limited to, infections caused by the following: Staphylococcus; Streptococcus, including S. pyogenes; Enterococci; Bacillus, such as, for example Bacillus anthracis, and Lactobacillus; Listeria; Corynebacterium diphtheriae; Gardnerella such as, for example G. vaginalis; Nocardia; Streptomyces; Thermoactinomyces vulgaris; Treponerna; Camplyobacter, Pseudomonas such as, for example, P. aeruginosa; Legionella; Neisseria such as, for example N. gonorrhoeae and N. meningitides; Flavobacterium such as, for example F. meningosepticum and F. odoraturn; Brucella; Bordetella such as, for example B. pertussis and B. bronchiseptica; Escherichia such as, for example E. coli, Klebsiella; Enterobacter, Serratia such as, for example S. marcescens and S. liquefaciens; Edwardsiella; Proteus such as, for example P. mirabilis and P. vulgaris; Streptobacillus; Rickettsiaceae such as, for example R. fickettsfi, Chlamydia such as, for example C. psittaci and C. trachornatis; Mycobacterium such as, for example M. tuberculosis, M. intracellulare, M. folluiturn, M. laprae, M. avium, M bovis, M. africanum, M. kansasii, and M. lepraernurium; and Nocardia.

Examples of protozoa infections that may be treated in the present invention include, but are not limited to, infections caused by leishmania, kokzidioa, and trypanosoma.

A complete list of infectious diseases can be found on the website of the National Center for Infectious Disease (NCID) at the Center for Disease Control (CDC) (World Wide Web (www) at cdc.gov/ncidod/diseases/), which list is incorporated herein by reference.

As used herein, the term “inflammatory diseases” includes a vast array of disorders and conditions that are characterized by inflammation.

Examples of inflammatory diseases include, but are not limited to, arthritis, rheumatoid arthritis, ankylosing spondylitis, osteoarthritis, psoriatic arthritis, juvenile idiopathic arthritis, juvenile rheumatoid arthritis, arthritis uratica, gout, chronic polyarthritis, periarthritis humeroscapularis, cervical arthritis, lumbosacral arthritis, enteropathic arthritis and ankylosing spondylitis, asthma, dermatitis, psoriasis, scleroderma, polymyositis, dermatomyositis, juvenila dermatomyositis, primary biliary cirrhosis, fibrosis, cystic fibrosis, pulmonary fibrosis, cirrhosis, endomyocardial fibrosis, dediastinal fibrosis, myelofibrosis, retroperitoneal fibrosis, nephrogenic fibrosis, Keloids, scleroderma, arthrofibrosis, post transplantation late and chronic solid organ rejection, multiple sclerosis, systemic lupus erythematosus, lupus nephritis, pemphigus, Pemphigus vulgaris, Pemphigus herpetiformis, Pemphigus vegetans, IgA pemphigus, Pemphigus erythematosus, bullous pemphigoid, Pemphigoid gestationis, Mucous membrane dermatosis, Pemphigoid nodularis, Linear IgA bullous dermatosis, Bullous lichen planus, Epidermolysis bullosa acquisita, autoimmune diabetes, diabetic retinopathy, diabetic nephropathy, diabetic vasculopathy, ocular inflammation, uveitis, rhinitis, ischemia-reperfusion injury, post-angioplasty restenosis, chronic obstructive pulmonary disease (COPD), glomerulonephritis, Graves disease, gastrointestinal allergies, conjunctivitis, atherosclerosis, coronary artery disease, angina, small artery disease, acute disseminated encephalomyelitis, idiopathic thrombocytopenic purpura, multiple sclerosis, systemic sclerosis, antiphospholipid syndrome, Sjoegren's syndrome, autoimmune hemolytic anemia, colitis, Crohn's Disease, ulcerative colitis, Inflammatory Bowel Disease (IBD), embolism, pulmonary embolism, arterial embolism, venous embolism, allergic inflammation, cardiovascular disease, graft-related diseases, graft versus host disease (GVHID), disorders associated with graft transplantation rejection, chronic rejection, and tissue or cell allografts or xenografts, autoimmune diseases, degeneration after trauma, stroke, transplant rejection, allergic conditions and hypersensitivity, e.g., allergic rhinitis, allergic eczema and the like, skin diseases, dermal inflammatory disorders, or any combination thereof.

As used herein, the term “autoimmune diseases” refers to diseases with defects in the immune system causing an immune response to self-tissue.

Examples of autoimmune diseases include, but are not limited to, lupus (e.g., lupus erythematosus, lupus nephritis), Hashimoto's thyroiditis, Wegener's disease; primary myxedema, Graves' disease, pernicious anemia, autoimmune atrophic gastritis, Addison's disease, diabetes (e.g. insulin dependent diabetes mellitus, type I diabetes mellitus, type II diabetes mellitus), good pasture's syndrome, myasthenia gravis, pemphigus, intestinal inflammatory conditions such as Crohn's disease and ulcerative colitis; sympathetic ophthalmia, autoimmune uveitis, multiple sclerosis, autoimmune hemolytic anemia, idiopathic thrombocytopenia, primary biliary cirrhosis, chronic action hepatitis, ulcerative colitis, Sjogren's syndrome, arthritis conditions such as rheumatoid arthritis, psoriatic arthritis, ankylosing spondylitis and juvenile idiopathic arthritis; polymyositis, scleroderma, psoriasis, primary sclerosing cholangitis; asthma, transplant rejection (host versus graft disease); graft versus host disease and mixed connective tissue disease.

As used herein, the term “Paroxysmal Nocturnal Hemoglobinuria” or “PNH” has its general meaning in the art and refers to an acquired clonal hematopoietic stem cell disorder characterized by corpuscular hemolytic anemia, bone marrow failure and frequent thrombotic events. In some embodiments, the subject is not mutated for the PIGA gene (phosphatidylinositol glycan anchor biosynthesis class A, Gene ID: 5277).

It will be understood that the treatment of the diseases, disorders or conditions may be mediated by different mechanisms, depending on the effector functions of the antibody or antigen-binding fragment thereof according to the present invention.

In one embodiment, the antibody or antigen-binding fragment thereof according to the present invention mediates ADCC, ADCP and/or CDC, allowing to target and deplete CD160-TM-expressing cells to which they are bound.

In one embodiment, administration of said antibody or fragment thereof may lead to the depletion of cells expressing CD160-TM (e.g., leads to a 10%, 20%, 30%, 40%, 50%, 60% or greater elimination or decrease in number of CD160-TM+NK cells), such as, for example CD160-TM expressing tumor cells.

As used herein, the term “deplete” with respect to a cell or a population of cells, refers to a measurable decrease in the number of said cells in the subject. The reduction can be at least about 10%, e.g., at least about 20%, 30%, 40%, 50%, 60%, 70%, 80%, 90%, 95%, 96%, 97%, 98%, 99%, or more. In some embodiments, the term refers to a decrease in the number of the cells in a subject or in a sample to an amount below detectable limits.

A further object of the present invention thus relates to a method of depleting a population of cells which express the CD160-TM isoform in a subject in need thereof, comprising delivering to the subject a therapeutically effective amount of an antibody or antigen-binding fragment thereof as described hereinabove, wherein said antibody or fragment thereof mediates ADCC, ADCP and/or CDC.

A further object of the present invention relates to a method of depleting a population of malignant NK cells which express the CD160-TM isoform in a subject in need thereof, comprising delivering to the subject a therapeutically effective amount of an antibody or antigen-binding fragment thereof as described hereinabove, wherein said antibody or fragment thereof mediates ADCC, ADCP and/or CDC.

A further object of the present invention relates to a method of depleting a population of cells which express the epitope recognized by the 21C8 or 22B12 antibody in a subject in need thereof, comprising delivering to the subject a therapeutically effective amount of an antibody or antigen-binding fragment thereof as described hereinabove, wherein said antibody or fragment thereof mediates ADCC, ADCP and/or CDC.

A further object of the present invention relates to a method of treating a cancer wherein cancer cells express CD160-TM in a subject in need thereof, comprising delivering to the subject a therapeutically effective amount of an antibody or antigen-binding fragment thereof as described hereinabove, wherein said antibody or fragment thereof mediates ADCC, ADCP and/or CDC.

A further object of the present invention relates to an antibody or antigen-binding fragment thereof as described hereinabove for use in treating a cancer in a subject in need thereof, wherein cancer cells express CD160-TM, wherein said antibody or fragment thereof mediates ADCC, ADCP and/or CDC.

In one embodiment, said cancer is a solid tumor. In one embodiment, said cancer is a liquid tumor, i.e. wherein cancer cells are present in the body fluids such as the blood or bone marrow.

Examples of cancers wherein cancer cells express CD160-TM include, but are not limited to, a NK leukemia or a NK lymphoma, such as for example, extranodal and non-extranodal NK/T lymphomas; NK cell derived malignancies; acute NK leukemia, and peripheral T-cell lymphoma (PTCL), such as for example peripheral T-cell lymphoma not otherwise specified (PTCL-NOS).

Examples of cancers wherein cancer cells express CD160-TM include, but are not limited to, breast cancers, such as, for example, luminal A breast cancer, luminal B breast cancer, triple-negative breast cancer (TNBC) and HER2-positive breast cancer.

Thus, in one embodiment, said cancer is a breast cancer. In one embodiment, the cancer is a breast cancer selected from the group comprising or consisting of luminal A breast cancer, luminal B breast cancer, triple-negative breast cancer (TNBC) and HER2-positive breast cancer.

In one embodiment, the cancer is luminal A breast cancer. In one embodiment, the cancer is luminal B breast cancer. In one embodiment, the cancer is TNBC. In one embodiment, the cancer is HER2-positive breast cancer.

Another object of the present invention is an antibody or fragment thereof binding to human CD160-TM for use in the treatment of a PTCL, such as for example peripheral T-cell lymphoma not otherwise specified (PTCL-NOS).

In one embodiment, the antibody or antigen-binding fragment thereof according to the present invention does not mediate ADCC, ADCP and/or CDC, allowing to target and activate CD160-TM-expressing cells to which they are bound.

In one embodiment, administration of said antibody or fragment thereof does not lead, directly or indirectly, to the depletion of NK cells expressing CD160-TM polypeptides (e.g., do not lead to a 10%, 20%, 30%, 40%, 50%, 60% or greater elimination or decrease in number of CD160-TM+NK cells).

In one embodiment, administration of said antibody or fragment thereof leads to an enhanced or improved NK cell activities or NK cell effector functions.

A further object of the present invention thus relates to a method of enhancing NK cell activities or NK cell effector functions, in particular NK cell killing activities in a subject in need thereof, comprising administering to the subject a therapeutically effective amount of an antibody or antigen-binding fragment thereof as described hereinabove, wherein said antibody or fragment thereof does not mediate ADCC, ADCP and/or CDC.

As used herein, “NK cells” refers to a sub-population of lymphocytes that is involved in innate or non-conventional immunity. NK cells can be identified by virtue of certain characteristics and biological properties, such as the expression of specific surface antigens including CD56 and/or CD16 for human NK cells, the absence of the alpha/beta or gamma/delta TCR complex on the cell surface, the ability to bind to and kill cells that fail to express “self” MHC/HLA antigens by the activation of specific cytolytic machinery, the ability to kill tumor cells or other diseased cells that express a ligand for NK activating receptors, and the ability to release protein molecules called cytokines that stimulate or inhibit the immune response (“NK cell activities”). Any subpopulation of NK cells will also be encompassed by the term NK cells. Within the context of this invention, “active” NK cells designate biologically active NK cells, including NK cells having the capacity of lysing target cells or enhancing the immune function of other cells. For instance, an “active” NK cell can be able to kill cells that express a ligand for an activating NK receptor and/or fail to express MHC/HLA antigens recognized by a KIR on the NK cell.

The ability of the antibody or antigen-binding fragment according to the present invention to enhance NK cell activities, in particular NK cell killing activities, may be determined by any assay well known in the art. Typically said assay is an in vitro assay, wherein NK cells are brought into contact with target cells (e.g., target cells that are recognized and/or lysed by NK cells). For example, the antibody or antigen-binding fragment can be selected for the ability to increase specific lysis by NK cells by more than about 20%, preferably with at least about 30%, at least about 40%, at least about 50%, or more of the specific lysis obtained at the same effector: target cell ratio with NK cells or NK cell lines that are contacted by the antibody or fragment thereof according to the present invention. Typically, NK cell cytotoxicity may be determined by any assay described in the Example part, such as i) an in vitro assay assessing the NK cells induced lysis of target cells using viability dye staining, or ii) an in vitro assay assessing the expression of NK cell activation markers, such CD107a expression. NK cell cytotoxicity may also be measured by a classical in vitro chromium release test of cytotoxicity. Effector cells may be fresh PB-NK from healthy donors or NK92 cell lines. The target cells may be the murine mastocytoma P815 cells, the EBV-infected B cell lines, or other lines such as K562 cells.

Accordingly, the antibody or antigen-binding fragment according to the present invention is selected if it causes an increase in the reactivity or cytoxicity of NK cells toward target cells (infected cells, tumor cells, pro-inflammatory cells, etc.), increased activation, activation markers (e.g., CD107 expression) and/or IFN gamma production in NK cells, and/or increased the frequency in vivo of such activated, reactive, cytotoxic and/or activated NK cells.

A further object of the present invention relates to an antibody or antigen-binding fragment thereof as described hereinabove, for use in treating a cancer in a subject in need thereof, wherein cancer cells do not express CD160-TM, wherein said antibody or fragment thereof does not mediate ADCC, ADCP and/or CDC.

A further object of the present invention relates to a method of treating a cancer wherein cancer cells do not express CD160-TM in a subject in need thereof, comprising delivering to the subject a therapeutically effective amount of an antibody or antigen-binding fragment thereof as described hereinabove, wherein said antibody or fragment thereof does not mediate ADCC, ADCP and/or CDC.

Examples of cancers are provided hereinabove.

A further object of the present invention relates to an antibody or antigen-binding fragment thereof as described hereinabove, for use in treating an infectious disease in a subject in need thereof, wherein said antibody or fragment thereof does not mediate ADCC, ADCP and/or CDC.

Examples of infectious diseases are provided hereinbelow.

A further object of the present invention relates to an antibody or antigen-binding fragment thereof as described hereinabove, for use in treating an inflammatory disease in a subject in need thereof, wherein said antibody or fragment thereof does not mediate ADCC, ADCP and/or CDC.

Examples of inflammatory diseases are provided hereinbelow.

A further object of the present invention relates to an antibody or antigen-binding fragment thereof as described hereinabove, for use in treating an autoimmune disease in a subject in need thereof, wherein said antibody or fragment thereof does not mediate ADCC, ADCP and/or CDC.

Examples of autoimmune diseases are provided hereinbelow.

A further object of the present invention relates to a method of treating an infectious disease, an inflammatory disease and/or an auto-immune disease in a subject in need thereof, comprising delivering to the subject a therapeutically effective amount of an antibody or antigen-binding fragment thereof as described hereinabove, wherein said antibody or fragment thereof does not mediate ADCC, ADCP and/or CDC.

A further object of the present invention relates to an antibody or antigen-binding fragment thereof as described hereinabove, for use in treating paroxysmal nocturnal hemoglobinuria (PNH) in a subject in need thereof, wherein said antibody or fragment thereof does not mediate ADCC, ADCP and/or CDC.

A further object of the present invention relates to a method of treating PNH in a subject in need thereof, comprising delivering to the subject a therapeutically effective amount of an antibody or antigen-binding fragment thereof as described hereinabove, wherein said antibody or fragment thereof does not mediate ADCC, ADCP and/or CDC.

In one embodiment, the antibody or antigen-binding fragment thereof, the fusion protein, the nucleic acid or the expression vector according to the present invention is the only active agent or therapeutic agent that is to be administered to the subject in need thereof.

In one embodiment, the antibody or antigen-binding fragment thereof, the fusion protein, the nucleic acid or the expression vector according to the present invention is used in combination with at least one further therapeutic agent.

In one embodiment, the administration of the at least one further therapeutic agent and of the antibody or antigen-binding fragment thereof, fusion protein, nucleic acid, or expression vector according to the present invention is simultaneous, separate or sequential.

In one embodiment, for simultaneous administration, the at least one further therapeutic agent and the antibody or antigen-binding fragment thereof, the fusion protein, the nucleic acid, or the expression vector according to the present invention are administered as one composition or as separate compositions, as appropriate.

In one embodiment, the at least one further therapeutic agent is a therapeutic agent useful for treating a disease, disorder or condition as described hereinabove.

Examples of additional therapeutic agents include, but are not limited to, chemotherapeutic agents, targeted cancer therapy, radiotherapy, immunotherapeutic agents or anti-cancer immunogens, anti-cancer antibodies, cytotoxic agents, anti-angiogenic agents, cell cycle control/apoptosis regulating agents, hormonal regulating agents, and other immunosuppressive and/or anti-inflammatory drugs selected from corticoids, such as, for example, glucocorticoids.

In one embodiment, the at least one further therapeutic agent is a natural ligand of an NK cell activating receptor or an antibody that binds and activates an NK cell activating receptor other than CD160-TM. In one embodiment, the agent is an agent that increases the presence of a natural ligand of an NK cell activating receptor on the surface of a target cell (e.g., infected cells, or tumor cells).

In one embodiment where the antibody or antigen-binding fragment thereof does not mediate ADCC, CDC and/or ADCP, said antibody or fragment thereof is used in combination with a further therapeutic agent that (i) is a natural ligand of an NK cell activating or an antibody that binds and activates an NK cell activating receptor other than CD160-TM and/or (ii) increases the presence of a natural ligand of an NK cell activating receptor on the surface of a target cell.

NK cell activating receptors include, for example, NKG2D or activating KIR receptors (KIR2DS receptors, KIR2DS2, KIR2DS4).

As used herein, the term “activating NK receptor” refers to any molecule on the surface of NK cells that, when stimulated, causes a measurable increase in any property or activity known in the art as associated with NK activity, such as cytokine (for example IFN-7 and TNF-α) production, increases in intracellular free calcium levels, the ability to target cells in a redirected killing assay as described, e.g., elsewhere in the present specification, or the ability to stimulate NK cell proliferation. The term “activating NK receptor” includes but is not limited to activating forms or KIR proteins (for example KIR2DS proteins), NKG2D, IL-2R, IL-12R, IL-15R, IL-18R and IL-21R. Examples of ligands that act as agonists at activating receptors include, e.g., IL-2, IL-15, IL-21 polypeptides.

In one embodiment, the at least one further therapeutic agent is an antibody specific for CD137. As used herein the term “CD137” has its general meaning in the art and may also be referred to as Ly63, ILA or 4-1BB. CD137 is a member of the tumor necrosis factor (TNF) receptor family. Members of this receptor family and their structurally related ligands are important regulators of a wide variety of physiologic processes and play an important role in the regulation of immune responses. CD137 is expressed by activated NK cells, T and B lymphocytes and monocytes/macrophages. The gene encodes a 255-amino acid protein with 3 cysteine-rich motifs in the extracellular domain (characteristic of this receptor family), a transmembrane region, and a short N-terminal cytoplasmic portion containing potential phosphorylation sites. Expression in primary cells is strictly activation dependent. The ligand for the receptor is TNFSF9. Human CD137 is reported to bind only to its ligand. Agonists include the native ligand (TNFSF9), aptamers (see McNamara et al. (2008) J. Clin. Invest. 1 18: 376-386), and antibodies.

In one embodiment, the at least one further therapeutic agent is an antibody which induces, via ADCC, the death of a cell expressing an antigen to which said antibody binds.

In one embodiment where the antibody or antigen binding fragment thereof according to the present invention does not mediate ADCC, ADCP and/or CDC, said antibody or fragment thereof is used in combination with an antibody which induces, via ADCC, the death of a cell expressing an antigen to which said antibody binds.

In one embodiment, said second antibody is specific for a cell surface antigen, e.g., membrane antigens.

In some embodiments, said second antibody is specific for a tumor antigen (e.g., molecules specifically expressed by tumor cells), such as CD20, CD52, ErbB2 (or HER2/Neu), CD33, CD22, CD25, MUC-1, CEA, KDR, aV03, etc., particularly lymphoma antigens (e.g., CD20).

Accordingly, the present invention also provides methods to enhance the antitumor effect of monoclonal antibodies directed against tumor antigen(s). In the methods of the invention, ADCC function is specifically augmented, which in turn enhances target cell killing, by sequential administration of an antibody directed against one or more tumor antigens, and an antibody or antigen-binding fragment according to the present invention.

Accordingly, a further object of the present invention relates to a method of enhancing NK cell ADCC of an antibody in a subject in need thereof, comprising administering to the subject said antibody, and administering to the subject an antibody or antigen-binding fragment according to the present invention, wherein preferably the antibody or fragment thereof according to the present invention does not mediate ADCC, ADCP and/or CDC.

A further object of the present invention relates to a method of treating cancer in a subject in need thereof, comprising administering to the subject a first antibody selective for a cancer cell antigen, and administering to the subject an antibody or antigen-binding fragment according to the present invention, wherein preferably the antibody or fragment thereof according to the present invention does not mediate ADCC, ADCP and/or CDC.

A number of antibodies are currently in clinical use for the treatment of cancer, and others are in varying stages of clinical development. Antibodies of interest for the methods of the invention act through ADCC, and are typically selective for tumor cells, although one of skill in the art will recognize that some clinically useful antibodies do act on non-tumor cells, e.g., CD20. There are a number of antigens and corresponding monoclonal antibodies for the treatment of B cell malignancies. One popular target antigen is CD20, which is found on B cell malignancies. Rituximab is a chimeric unconjugated monoclonal antibody directed at the CD20 antigen. CD20 has an important functional role in B cell activation, proliferation, and differentiation. The CD52 antigen is targeted by the monoclonal antibody alemtuzumab, which is indicated for treatment of chronic lymphocytic leukemia. CD22 is targeted by a number of antibodies, and has recently demonstrated efficacy combined with toxin in chemotherapy-resistant hairy cell leukemia. Monoclonal antibodies targeting CD20, also include tositumomab and ibritumomab. Monoclonal antibodies useful in the methods of the invention, which have been used in solid tumors, include without limitation edrecolomab and trastuzumab (herceptin). Edrecolomab targets the 17-1 A antigen seen in colon and rectal cancer, and has been approved for use in Europe for these indications. Its antitumor effects are mediated through ADCC, CDC, and the induction of an anti-idiotypic network. Trastuzumab targets the HER-2/neu antigen. This antigen is seen on 25% to 35% of breast cancers. Trastuzumab is thought to work in a variety of ways: downregulation of HER-2 receptor expression, inhibition of proliferation of human tumor cells that overexpress HER-2 protein, enhancing immune recruitment and ADCC against tumor cells that overexpress HER-2 protein, and downregulation of angiogenesis factors. Alemtuzumab (Campath) is used in the treatment of chronic lymphocytic leukemia; colon cancer and lung cancer; Gemtuzumab (Mylotarg) finds use in the treatment of acute myelogenous leukemia; Ibritumomab (Zevalin) finds use in the treatment of non-Hodgkin's lymphoma; Panitumumab (Vectibix) finds use in the treatment of colon cancer. Cetuximab (Erbitux) is also of interest for use in the methods of the invention. The antibody binds to the EGF receptor (EGFR), and has been used in the treatment of solid tumors including colon cancer and squamous cell carcinoma of the head and neck (SCCHN).

In one embodiment, the at least one further therapeutic agent is an immune checkpoint inhibitor (ICI).

Various tumors are able to express molecular factors protecting them from being attacked by the immune system, and are thus capable of successfully escaping the immune system supervision control. This “tumor immune escape” is mainly due to the antagonistic blocking of receptors and binding sites targeted by immune cell ligands. Immune checkpoint inhibitors are molecules especially targeting this kind of inhibitory mechanisms developed by tumorous cells.

Examples of ICIs include, but are not limited to, inhibitors of CTLA-4 (such as, for example, ipilumab and tremelimumab), inhibitors of PD-1 (such as, for example, pembrolizumab, pidilizumab, nivolumab and AMP-224) inhibitors of PD-L1 (such as, for example, atezolizumab, avelumab, durvalumab and BMS-936559), inhibitors of LAG3 (such as, for example, IMP321) and inhibitors of B7-H3 (such as, for example, MGA271).

In one embodiment where the antibody or antigen-binding fragment thereof according to the present invention does not mediates ADCC, ADCP and/or CDC, said antibody or fragment thereof is used in combination with at least one immune checkpoint inhibitor (ICI).

The present invention also relates to the use of at least one antibody or antigen-binding fragment thereof according to the present invention in detecting CD160-TM in a sample, preferably in a biological sample, in vitro or in vivo. The present invention also relates to the use of at least one antibody or antigen-binding fragment thereof according to the present invention in detecting and/or quantifying CD160-TM in a sample, preferably in a biological sample, in vitro or in vivo.

The present invention further relates to an antibody or antigen-binding fragment thereof according to the present invention for use in the in vivo detection of tumors expressing CD160-TM in a subject, preferably wherein the antibody or fragment thereof is conjugated with a detectable label. Examples of cancers wherein cancer cells express CD160-TM are provided hereinabove. Examples of detectable labels are provided hereinabove.

The present invention further relates to an in vitro method for detecting CD160-TM in a sample, preferably in a biological sample, comprising a step of contacting the sample with at least one antibody or antigen-binding fragment thereof as described hereinabove.

Examples of assays in which the antibody or antigen-binding fragment thereof according to the present invention may be used, include, but are not limited to, ELISA, sandwich ELISA, radioimmunoassay (RIA), fluorescence-activated cell sorting (FACS), tissue immunohistochemistry, Western-blot, and immunoprecipitation.

The present invention further relates to a method for detecting CD160-TM in a sample, comprising a step of contacting the sample with an antibody or antigen-binding fragment thereof according to the present invention and a step of detecting the anti-CD160-TM antibody or fragment thereof bound to CD160-TM, thereby indicating the presence of CD160-TM in the sample. In one embodiment, said method is an in vitro method.

In one embodiment, the sample is a biological sample. Examples of biological samples include, but are not limited to, bodily fluids, preferably blood, more preferably blood serum, plasma, synovial fluid, bronchoalveolar lavage fluid, sputum, lymph, ascitic fluids, urine, amniotic fluid, peritoneal fluid, cerebrospinal fluid, pleural fluid, pericardial fluid, and alveolar macrophages, tissue lysates and extracts prepared from diseased tissues.

In one embodiment, the sample is abreast tumor tissue. In one embodiment, said breast tumor tissue is from luminal A breast cancer, luminal B breast cancer, HER2 positive breast cancer, and TNBC.

In one embodiment, the antibody or antigen-binding fragment thereof according to the present invention is conjugated with a detectable label and may be detected directly. Example of detectable labels are provided hereinabove.

In one embodiment, the antibody or antigen-binding fragment thereof according the present invention is unlabeled (and is referred as the first/primary antibody) and is used in combination with a secondary antibody or other molecule that can bind the anti-CD160-TM antibody and is labeled. Examples of detectable labels that may be bind to said secondary antibody or other molecule include, without limitation, the detectable labels as provided hereinabove.

As compared to the anti-CD160-TM antibodies of the prior art, the antibodies or antigen-binding fragments according to the present invention may present one or several of the following advantages:
In some embodiments, the antibody or antigen-binding fragment according to the present invention does not need secondary antibodies to elicit ADCC, CDC, and/or ADCP;
In some embodiments, the antibody or antigen-binding fragment according to the present invention presents an increased ADCC, CDC, and/or ADCP, as compared to the CD160-TM antibodies of the prior art;
In some embodiments, the antibody or antigen-binding fragment according to the present invention presents increased capacities of NK cell activation or induces increased NK cell induced lysis, as compared to the CD160-TM antibodies of the prior art;
In some embodiments, the antibody or antigen-binding fragment according to the present invention is suitable for therapeutic use, such as, for example, in humans;
In some embodiment, the antibody or antigen-binding fragment thereof according to the present invention presents an increased affinity for CD160-TM, in particular an increased affinity for CD160-TM in ELISA tests, as compared to the CD160-TM antibodies of the prior art.
In some embodiment, the antibody or antigen-binding fragment according to the present invention allows detection of CD160-TM in a non-native form, such as, for example, on fixed samples or tissues; and may thus be used for immunohistochemical or immunofluorescence applications.

### BRIEF DESCRIPTION OF THE DRAWINGS

FIG. 1 is a combination of two graphs showing K562 target cell lysis, indicated by the percentage of 7AAD positive cells, induced by NK92 cells in presence of a control murine immunoglobulin G isotype 2a (muIgG2a) or the anti-CD160-TM antibodies of the present invention (22B12 and 21C8 antibodies) at a ratio of effector (E) and target (T) cells of 8/1. FIG. 1A represents the percentage of 7AAD positive cells among K562 cells in presence of a control murine IgG2a antibody or 21C8 antibody at the E/T cells ratio of 8/1. n=4 per group, *p<0.05 vs muIgG2a, Paired t-test. FIG. 1B represents the percentage of 7AAD positive cells among K562 cells in presence of a control murine IgG2a antibody or 22B12 antibody at the E/T cells ratio of 8/1. n=4 per group, *p<0.05 vs muIgG2a, Paired t-test.

FIG. 2 is a histogram showing the percentage of NK cells expressing CD107a, a marker of NK cell degranulation, in the presence of the anti-CD160-TM antibodies of the present invention (22B12 and 21C8) and Raji cells at different E/T ratios (2.5/1, 5/1, 10/1, 20/1). Results are expressed as Mean ±SEM (Ab-control isotype) (n=3 donors).

FIG. 3 is a histogram showing the degranulation of NK cells when exerting ADCC towards a CD160-negative target cell (Raji cells) or a CD160-positive target cell (Raji cells expressing CD160-TM) in the presence of 21C8 antibody. Results are expressed as Mean ±SEM (Ab-control isotype) (n=3), *p<0.05 vs Raji, Unpaired t-test.

FIG. 4 is graph showing the induction of ADCC on spheroids of IJG1731 breast cancer cell line expressing GFP, monitored by the surface of green area, in response to the addition of an anti CD160-TM antibody of the present invention (22B12 antibody) or an isotype control antibody (murine IgG2a).

FIG. 5 is a combination of three histograms showing the induction of ADCP, indicated by the percentage of phagocytosis, in presence of an anti CD160-TM antibody of the present invention (22B12 antibody) at 1 or 10 μg/mL, or an isotype control antibody (murine IgG2a antibody) in three breast cancer cell lines: IJG1731 (A), MCF-7 (B), and MDA-MB-231 (C.

FIG. 6 is a combination of immunofluorescence images with the anti CD160-TM antibody 22B12 or an isotype control antibody (murine IgG2a antibody) in three TNBC cell lines: IJG1731, MDA-MB-231, and BT20, and one luminal A cancer cell line: MCF7.

FIG. 7 is a combination of two histograms showing the percentage of CD160-TM expressing tumor cells (A) and the staining intensity (B) with an anti-CD160-TM antibody of the present invention (22B12 antibody) in breast tumor tissues from various cancers: HER2 positive cancer, TNBC, luminal A and luminal B.

FIG. 8 is a combination of a schema (A) and a graph (B) showing the effect of the administration of an anti-CD160-TM antibody of the present invention (21C8 antibody) on tumor volume in a breast tumor mouse model, as compared to a control murine IgG2a (muIgG2a). FIG. 8A represents a schema of the protocol used, wherein SCID mice are injected intra-mammary with BT-20 cells, and treated at days 10, 13, 17 and 19 with either a control murine IgG2a (muIgG2a) or the anti-CD160-TM 21C8 antibody. FIG. 8B represents the tumor volume monitored on days 10 to 20 in mice treated with either the muIgG2a or the anti-CD160-TM 21C8 antibody. n=6 per group *p<0,05, **p<0,01, ***p<0,001, ****p<0,0001 vs isotype control, Two-way ANOVA

### EXAMPLES

The present invention is further illustrated by the following examples.

### Materials and Methods

### NK Cell Activation

NK92 cells (effector cells) were pre-incubated with either murine IgG2a control isotype, CD160-TM 21C8 antibody or CD160-TM 22B12 antibody (10 μg/ml). Cells were then incubated with K562 cells (target cells) at a ratio of E/T cells of 8/1 for 4h30 at 37° C.

Labelings were performed with CD56-PE and 7AAD apoptosis dye staining. Cells were analyzed by flow cytometry and results are expressed as % of 7AAD+ cells among K562; Mean±SEM (n=4 experiments).

### CD107a Analysis

PBMC were used as source of NK cells. To evaluate the gain of degranulation induced by the engagement of CD160-TM on NK cells, PBMC were mixed with Raji cells at E/T ratios of 2.5/1, 5/1, 10/1 or 20/1 (to induce CD160-TM expression on NK cells) in the presence of either murine IgG2a control isotype, CD160-TM 21C8 antibody or CD160-TM 22B12 antibody (10 μg/ml). After 24h of incubation at 37° C., labelings were performed with a mix of anti-CD3, -CD19, -CD56 and -CD107a and expression of CD107a on gated CD3⁻CD56⁺ NK cells was assessed by flow cytometry. Similar experiments were conducted using Raji cells forced to express CD160-TM (Raji-CD160-TM) to evaluate the degranulation of NK cells when displaying ADCC.

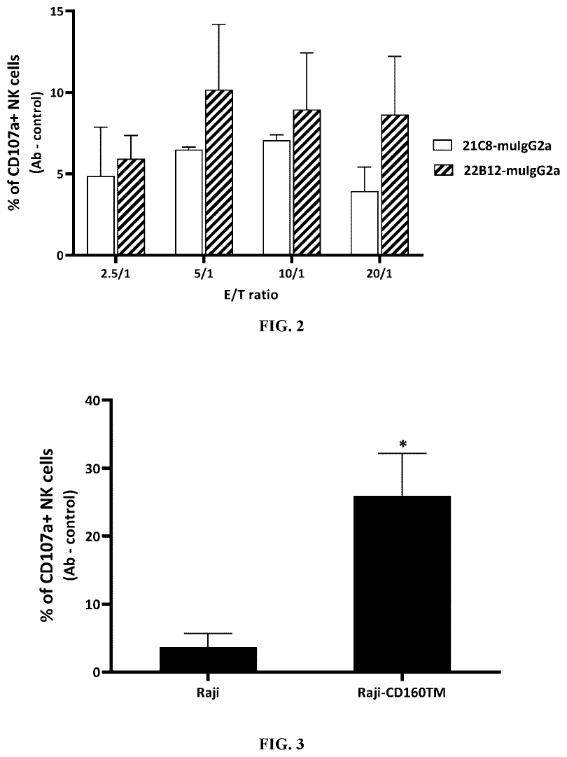

Results of FIG. 2 are expressed as the gain of degranulation induced by CD160-TM antibodies (21C8 or 22B12) as compared to the degranulation induced by the IgG2a control isotype (% with Ab-% with control), depending on the E/T ratio.

### Immunofluorescence on Breast Cancer Cell Lines

After adhesion on glass slides, breast cancer cell lines were fixed in PBS/4% paraformaldehyde. After a blocking step in PBS/3% goat serum/3% BSA, anti-CD160-TM antibody 22B12 or control isotype (muIgG2a) was added. After washes, AlexaFluor 594-coupled anti-mouse Igs antibodies (Invitrogen) were added. Slides were mounted with a DAPI Fluoromount solution (Southern Biotech).

### Antibody-Dependent Cellular Cytotoxicity (ADCC)

TNBC IJG1731 cell line was stably transfected with GFP. For spheroids formation, cells were plated in ultra-low adherence 96-well plates in culture medium supplemented with 2.5% Matrigel. After 96h, PBMC were added as source of NK cells, together with a mixture of IL2 and IL15 (to promote activation) and either anti-CD160-TM antibody 22B12 or control isotype (muIgG2a). Live cell imaging was the performed to monitor the green fluorescence associated to the spheroids (acquisition of one image each 6 hours). Results are expressed as Mean ±SD of the surface of green fluorescence from triplicates.

### Antibody-Dependent Cellular Phagocytosis (ADCP)

Breast cancer cell lines were loaded with CFSE and then incubated with the CD33+ THP-1 monocytic cell line at various E/T ratios, in the presence of anti-CD160-TM or control Ab (1 or 10 mg/mL) for 2h30 at 37° C. ADCP was assessed by flow cytometry by analyzing the transfer of CFSE staining to THP-1 cells (detection of CD33+ CFSE+ cells).

### In Vivo Effect of CD160-TM Depletion

Eight-week old female SCID mice were injected intra-mammary with 5×10⁶ CD160-TM-positive triple-negative breast cancer cell line BT-20. When the tumors reached approximately 100 mm³ (day 10), mice were randomized in 2 groups of 6 and antibody treatment started the day after. 21C8 or isotype-matched negative control (muIgG2a) were administrated intra-peritoneally (100 μg) twice weekly. Tumor size was measured at each antibody injection day (day 10, 13, 17 and 19) and at sacrifice (day 20).

### Results

### Functionality of the CD160-TM Antibodies

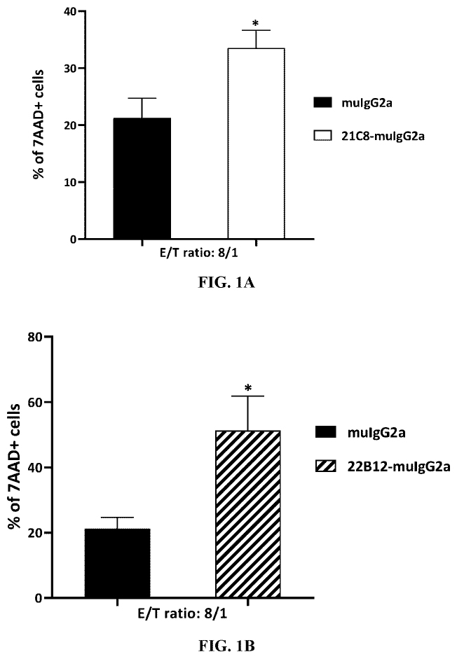

The ability of the anti-CD160-TM antibodies of the present invention to increase NK cell natural cytotoxicity was first evaluated. FIGS. 1A-B show that both antibodies 21C8 and 22B12 of the present invention are able to target CD160-TM and consequently to promote an increased NK cell killing activity against target cells, as compared to a control murine antibody.

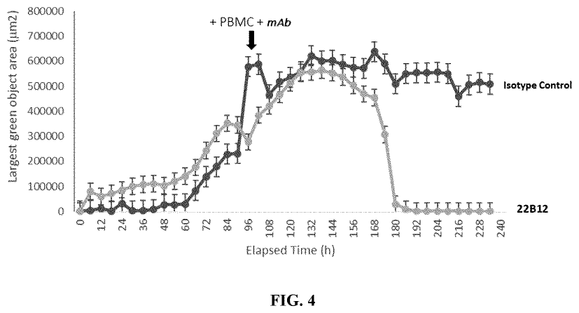

In addition, the expression of CD107a, a marker of NK cell degranulation, was assessed in presence of the CD160-TM antibodies of the present invention (21C8 and 22B12 antibody). As shown in FIGS. 2 and 3, the anti-CD160-TM antibodies of the present invention are able to positively modulate NK cell degranulation when used either as agonist antibodies on the effector cells (FIG. 2) or as depleting antibodies towards CD160-TM-expressing target cells (FIG. 3).

The ability of the anti CD160-TM antibodies of the present invention (22B12 antibody) to induce ADCC was further evaluated on spheroids from GFP-expressing IJG1731 cell line. As shown in FIG. 4, the addition of the 22B12 antibody induces a decrease in the fluorescence associated to the spheroids, indicative of the induction of ADCC by the 22B12 antibody, while a control isotype antibody is unable to promote GFP-positive cells' disappearance.

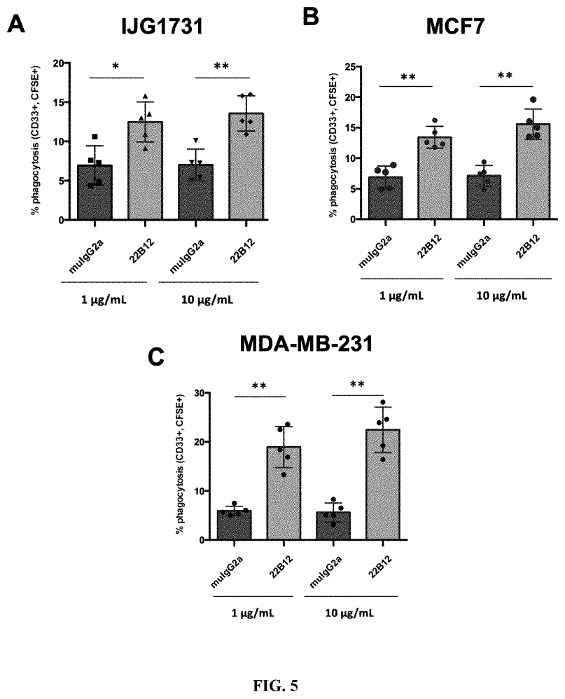

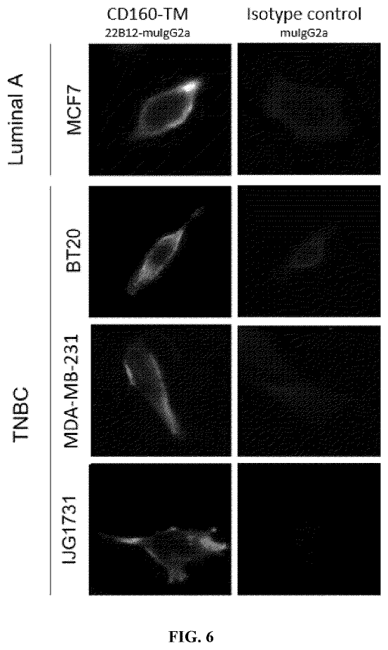

The ability of the anti CD160-TM antibodies of the present invention (22B12 antibody) to induce ADCP was also evaluated on 3 breast cancer cell lines. As shown in FIGS. 5A-5C, the presence of the 22B12 antibody increases the phagocytosis of breast cancer cell lines as compared to a control isotype antibody.

Taken together, these results clearly indicate that the anti-CD160-TM antibodies of the present invention can increase NK cell cytotoxic activity and promote both ADCC and ADCP, confirming the fact that these antibodies can be used in human therapy.

### Detection of CD160-TM in Cancer

The ability of the CD160-TM antibodies of the present invention to detect cancer cells expressing CD160-TM was then evaluated.

FIG. 6 shows that the anti-CD160-TM antibodies of the present invention (22B12 antibody) can detect CD160-TM in various breast cancer cell lines, including TNBC and luminal A cancer cell lines.

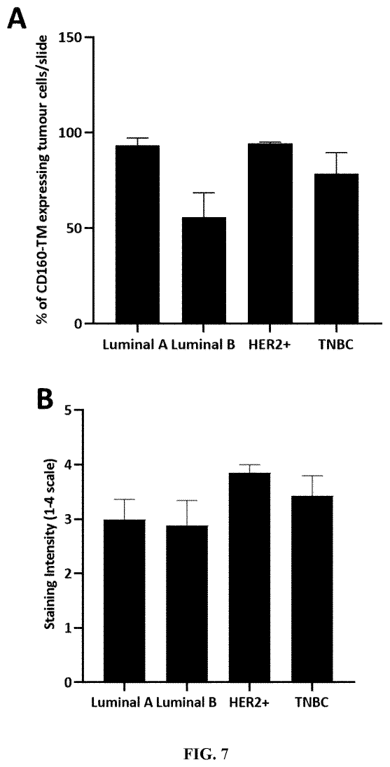

As shown in FIGS. 7A and 7B, CD160-TM expressing tumor cells were detected with an anti CD160-TM antibody of the present invention (22B12 antibody) in breast cancer tissues, including tissues from luminal A breast cancer, luminal B breast cancer, HER2 positive breast cancer, and TNBC.

Altogether, these data show that various subtypes of breast cancers are constituted of CD160-TM positive tumor cells, and that the CD160-TM antibodies of the present invention can be used to detect the presence of those CD160-TM positive tumor cells.

### In Vivo Effect of CD160-TM Depletion

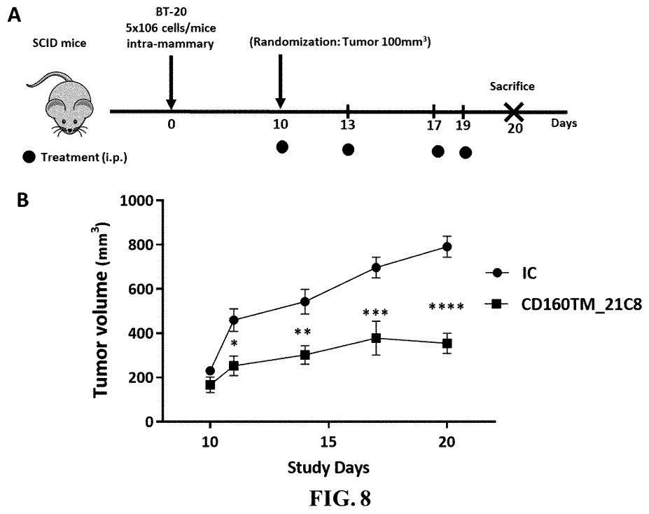

The ability of the anti-CD160-TM antibodies of the present invention to treat cancer was evaluated in vivo in a breast cancer mouse model. FIGS. 8A-B demonstrate that the administration of an anti-CD160-TM antibody of the present invention, namely 21C8 antibody, decreases the tumor growth as compared to a murine IgG2a control isotype.

These data clearly support the use of the anti-CD160-TM antibodies of the present invention to treat cancer, such as, for example, breast cancers.

1. An isolated anti-human CD160-TM antibody or antigen-binding fragment thereof, wherein the variable region of the light chain (V_{L}) of said antibody or antigen-binding fragment comprises the following complementary-determining region 3 (CDR3): X₁QSX₂SYPX₃T (SEQ ID NO: 15) wherein X₁ is G or L, X₂ is Y or Q, and X₃ is Y or F.

2. The isolated anti-human CD160-TM antibody or antigen-binding fragment thereof according to claim 1, wherein the variable region of the heavy chain (V_{H}) comprises the three following complementary-determining regions (CDRs):
CDR1:
(SEQ ID NO: 1)
GYTFTSYW,
CDR2:
(SEQ ID NO: 2)
IYPGSGFT,
CDR3:
(SEQ ID NO: 3)
SLDEGFAY;
wherein the variable region of the light chain (V_{L}) comprises the three following CDRs:
CDR1:
(SEQ ID NO: 4)
QGINIW,
CDR2:
KAS,
CDR3:
(SEQ ID NO: 5)
LQSQSYPFT.

3. The isolated anti-human CD160-TM antibody or antigen-binding fragment thereof according to claim 1, wherein the variable region of the heavy chain (V_{H}) comprises the three following complementary-determining regions (CDRs):
CDR1:
(SEQ ID NO: 6)
GFSLTSYG,
CDR2:
(SEQ ID NO: 7)
IWRGGNT,
CDR3:
(SEQ ID NO: 8)
VKNGGNSKDYALDY;
wherein the variable region of the light chain (V_{L}) comprises the three following CDRs:
CDR1:
(SEQ ID NO: 9)
ENVGIY;
CDR2:
GSS,
CDR3:
(SEQ ID NO: 10)
GQSYSYPYT.

4. The isolated anti-human CD160-TM antibody or antigen-binding fragment thereof according to claim 1, wherein said antibody or antigen-binding fragment comprises a heavy chain variable region comprising a sequence having at least 70% of identity with SEQ ID NO: 11 and a light chain variable region comprising a sequence having at least 70% of identity with SEQ ID NO: 12.

5. The isolated anti-human CD160-TM antibody or antigen-binding fragment thereof according to claim 1, wherein said antibody or antigen-binding fragment comprises a heavy chain variable region comprising a sequence having at least 70% of identity with SEQ ID NO: 13 and a light chain comprising a sequence having at least 70% of identity with SEQ ID NO: 14.

6. The isolated anti-human CD160-TM antibody or antigen-binding fragment thereof according to claim 1, which is chimeric or humanized, preferably wherein said antibody or antigen-binding fragment thereof is monoclonal.

7. The isolated anti-human CD160-TM antibody or antigen-binding fragment thereof according to claim 1, being a bispecific antibody.

8. The isolated anti-human CD160-TM antibody or antigen-binding fragment thereof according to claim 1, being conjugated, preferably wherein said antibody or fragment thereof is conjugated to a detectable label.

9. A fusion protein comprising the isolated anti-human CD160-TM antibody or the antigen-binding fragment thereof according to claim 1.

10. A nucleic acid encoding the isolated anti-human CD160-TM antibody or antigen-binding fragment thereof according to claim 1.

11. A pharmaceutical composition comprising the isolated anti-human CD160-TM antibody or antigen-binding fragment thereof according to claim 1, and at least one pharmaceutically acceptable excipient.

12. (canceled)

13. A method for the treatment of a cancer, an infectious disease, an autoimmune disease, an inflammatory disease or Paroxysmal Nocturnal Hemoglobinuria, said method comprising a step of administering an effective amount of an isolated anti-human CD160-TM antibody or antigen-binding fragment thereof according to claim 1 to a subject in need thereof.

14. An in vitro method for detecting CD160-TM in a sample, preferably in a biological sample, comprising a step of contacting the sample with at least one isolated anti-human CD160-TM antibody or antigen-binding fragment thereof according to claim 1.

15. A method for the in vivo detection of tumors expressing CD160-TM in a subject, comprising a step of administering an isolated anti-human CD160-TM antibody or antigen-binding fragment thereof according to claim 1 to a subject.

16. A nucleic acid encoding a fusion protein according to claim 9.

17. A pharmaceutical composition comprising the fusion protein according to claim 9 and at least one pharmaceutically acceptable excipient.

18. A method for the treatment of a cancer, an infectious disease, an autoimmune disease, an inflammatory disease or Paroxysmal Nocturnal Hemoglobinuria, said method comprising a step of administering an effective amount of the fusion protein according to claim 9 in a subject in need thereof.

19. A method for the treatment of a cancer, an infectious disease, an autoimmune disease, an inflammatory disease or Paroxysmal Nocturnal Hemoglobinuria, said method comprising a step of administering an effective amount of the pharmaceutical composition according to claim 11 in a subject in need thereof.


SEQUENCE LISTING (22 sequences)

SEQ ID NO: 1 - protein, 8 aa, synthetic construct
  REGION 1..8: note = VH-CDR1
GYTFTSYW

SEQ ID NO: 2 - protein, 8 aa, synthetic construct
  REGION 1..8: note = VH-CDR2
IYPGSGFT

SEQ ID NO: 3 - protein, 8 aa, synthetic construct
  REGION 1..8: note = VH-CDR3
SLDEGFAY

SEQ ID NO: 4 - protein, 6 aa, synthetic construct
  REGION 1..6: note = VL-CDR1
QGINIW

SEQ ID NO: 5 - protein, 9 aa, synthetic construct
  REGION 1..9: note = VL-CDR3
LQSQSYPFT

SEQ ID NO: 6 - protein, 8 aa, synthetic construct
  REGION 1..8: note = VH-CDR1
GFSLTSYG

SEQ ID NO: 7 - protein, 7 aa, synthetic construct
  REGION 1..7: note = VH-CDR2
IWRGGNT

SEQ ID NO: 8 - protein, 14 aa, synthetic construct
  REGION 1..14: note = VH-CDR3
VKNGGNSKDYALDY

SEQ ID NO: 9 - protein, 6 aa, synthetic construct
  REGION 1..6: note = VL-CDR1
ENVGIY

SEQ ID NO: 10 - protein, 9 aa, synthetic construct
  REGION 1..9: note = VL-CDR3
GQSYSYPYT

SEQ ID NO: 11 - protein, 115 aa, synthetic construct
  REGION 1..115: note = VH
QVQLQQPGSELVRPGASVRLSCKASGYTFTSYWMHWVRQRHGQGLEWIGNIYPGSGFTNYDEKFKNKGTLTVDTSSSTAYMHLSSLTSEDSAVYYCSLDEGFAYWGQGTLVTVSA

SEQ ID NO: 12 - protein, 107 aa, synthetic construct
  REGION 1..107: note = VL
DIQMNQSPSSLSASLGDTITITCRASQGINIWLNWYQQKPGNIPKRLIYKASNLHTGVPPRFRGSGSGTDFTLTISSLQPEDIATYYCLQSQSYPFTFGSGTKLEIR

SEQ ID NO: 13 - protein, 120 aa, synthetic construct
  REGION 1..120: note = VH
QVQLKQSGPGLVQPSQSLSIMCTVSGFSLTSYGLHWVRQSPGKGLEWLGVIWRGGNTDYNADFMSRLTITKDNSKSQVFFKMNSLQADDTAVYYCVKNGGNSKDYALDYWGQGTSVTVSS

SEQ ID NO: 14 - protein, 107 aa, synthetic construct
  REGION 1..107: note = VL
NIVMTQSPKSMSMSVGERVTLSCKASENVGIYVSWYQQKPEQSPKLLIYGSSNRYTGVPDRFTGSGSATDFTLIISSVQAEDLADYHCGQSYSYPYTFGGGTKLEIK

SEQ ID NO: 15 - protein, 9 aa, synthetic construct
  SITE 1: note = X is G or L
  REGION 1..9: note = VL-CDR3
  SITE 4: note = X is Y or Q
  SITE 8: note = X is Y or F
XQSXSYPXT

SEQ ID NO: 16 - protein, 8 aa, synthetic construct
  REGION 1..8: note = VH-CDR1
  SITE 2: note = X is F or Y
  SITE 3: note = X is S or T
  SITE 4: note = X is L or F
  SITE 8: note = X is G or W
GXXXTSYX

SEQ ID NO: 17 - DNA, 345 bp, synthetic construct
  misc_feature 1..345: note = VH
  source 1..345: mol_type = other DNA
caggtccaactgcagcaacctgggtctgagctggtgaggcctggagcttcagtgaggctgtcctgcaaggcttctggctacacattcaccagctactggatgcactgggtgaggcagaggcatggacaaggccttgagtggattggaaatatttatcctggtagtggttttactaattacgatgagaaattcaagaacaagggcacactgactgtagacacatcctccagtacagcctacatgcacctcagcagcctgacatctgaggactctgcggtctattactgttcattggacgaagggtttgcttactggggccaagggactctggtcactgtctctgca

SEQ ID NO: 18 - DNA, 321 bp, synthetic construct
  misc_feature 1..321: note = VL
  source 1..321: mol_type = other DNA
gacatccaaatgaaccagtctccatccagtctgtctgcatccctcggagacacaattaccatcacttgccgtgccagtcagggcattaatatttggttaaactggtaccagcagaaaccaggaaatattcctaaacgattgatctataaggcttccaacttgcacacaggagtcccaccaaggtttagaggcagtggatctggaacagatttcacattaactatcagcagcctacagcctgaagacattgccacttactactgtctacagagtcaaagttatccattcacgttcggctcggggacaaagttggaaattaga

SEQ ID NO: 19 - DNA, 360 bp, synthetic construct
  misc_feature 1..360: note = VH
  source 1..360: mol_type = other DNA
caggtgcagctgaagcagtcaggacctggcctagtgcagccctcacagagcctgtccataatgtgcacagtctctggtttctcattaactagctatggtttacactgggttcgccagtctccaggaaagggtctggagtggctgggagtgatatggagaggtggaaacacagactacaatgcagatttcatgtccagactgaccatcaccaaggacaattccaagagccaagttttctttaaaatgaacagtctgcaagctgatgacactgccgtttactactgtgtcaaaaatggcggtaactccaaggactatgctttggactactggggtcaaggaacctcagtcaccgtctcctca

SEQ ID NO: 20 - DNA, 321 bp, synthetic construct
  misc_feature 1..321: note = VL
  source 1..321: mol_type = other DNA
aacattgtaatgacccaatctcccaaatccatgtccatgtcagtaggagagagggtcaccttgagctgcaaggccagtgagaatgtgggtatttatgtatcctggtatcaacagaaaccagagcagtctcctaaactgctgatatacgggtcatccaaccggtacactggggtccctgatcgcttcacaggcagtggatctgcaacagatttcactctgatcatcagcagtgttcaggctgaagaccttgcagattatcactgtggacagagttacagttatccgtacacgttcggaggggggaccaagctggaaataaaa

SEQ ID NO: 21 - protein, 234 aa, Homo sapiens
MLLEPGRGCCALAILLAIVDIQSGGCINITSSASQEGTRLNLICTVWHKKEEAEGFVVFLCKDRSGDCSPETSLKQLRLKRDPGIDGVGEISSQLMFTISQVTPLHSGTYQCCARSQKSGIRLQGHFFSILFTETGNYTVTGLKQRQHLEFSHNEGTLSSGFLQEKVWVMLVTSLVALQGMSKRAVSTPSNEGAIIFLPPWLFSRRRRLERMSRGREKCYSSPGYPQESSNQFH

SEQ ID NO: 22 - protein, 160 aa, Homo sapiens
MLLEPGRGCCALAILLAIVDIQSGGCINITSSASQEGTRLNLICTVWHKKEEAEGFVVFLCKDRSGDCSPETSLKQLRLKRDPGIDGVGEISSQLMFTISQVTPLHSGTYQCCARSQKSGIRLQGHFFSILFTETGNYTVTGLKQRQHLEFSHNEGTLSS